(19) United States
(12) Patent Application Publication
(10) Pub. No.: US 2025/0302935 A1
(43) Pub. Date: Oct. 2, 2025

(54) DETECTION OF A GENETIC FUSION OR DELETION THAT RESULTS IN EXPRESSION OF A NEOANTIGEN

(71) Applicant: Flagship Pioneering Innovations VI, LLC, Cambridge, MA (US)

(72) Inventor: Anthony P. Shuber, Cambridge, MA (US)

(21) Appl. No.: 19/067,594

(22) Filed: Feb. 28, 2025

Related U.S. Application Data
(63) Continuation of application No. PCT/US23/73113, filed on Aug. 29, 2023.
(60) Provisional application No. 63/402,053, filed on Aug. 29, 2022.
(60) Provisional application No. 63/432,009, filed on Dec. 12, 2022.

(51) Int. Cl. A61K 39/00; C07K 14/47; C12Q 1/6886

(52) U.S. Cl. CPC A61K 39/001196; C07K 14/4748; C12Q 1/6886; C07K 2319/00; C12Q 2600/154

(57) ABSTRACT

The invention provides methods of detecting a sequence modification (e.g., a genetic fusion or deletion) associated with cancer development that results in expression of a neoantigen. The neoepitope serves as the basis for manufacture of a vaccine, which is administered to a subject to induce an immune response against those cells producing the neoantigen.

### RELATED APPLICATIONS

This application claims the benefit of and priority to U.S. Provisional Application No. 63/402,053, filed Aug. 29, 2022 and U.S. Provisional Application No. 63/432,009, filed Dec. 12, 2022, the contents of each of which are incorporated by reference herein in their entirety.

### FIELD OF THE INVENTION

The disclosure relates generally to methods of detecting a sequence modification (e.g., a genetic fusion or deletion) associated with cancer development that results in expression of a neoantigen. The neoepitope serves as the basis for manufacture of a vaccine, which is administered to a subject to induce an immune response against those cells producing the neoantigen.

### BACKGROUND

Cancer immunotherapy (e.g., cancer vaccine) has emerged as a promising cancer treatment modality. The goal of cancer immunotherapy is to harness the immune system for selective destruction of cancer while leaving normal tissues unharmed. Traditional cancer vaccines typically target tumor-associated antigens. Tumor-associated antigens are typically present in normal tissues, but overexpressed in cancer. However, because these antigens are often present in normal tissues, immune tolerance can prevent immune activation.

Neoantigens represent an attractive target for cancer immunotherapies. Neoantigens are derived from somatic mutations in the tumor cell genome and are not expressed on the surface of normal cells. Accordingly, cancer vaccines targeting cancer neoantigens have potential advantages, including decreased central immune tolerance and an improved safety profile.

The mutational landscape of cancer is complex and tumor mutations are generally unique to each individual subject. Most somatic mutations detected by sequencing do not result in effective neoantigens. Only a small percentage of mutations in the tumor DNA, or a tumor cell, are transcribed, translated, and processed into a tumor-specific neoantigen with sufficient accuracy to design a vaccine that is likely to be effective. Further, not all neoantigens are immunogenic. Moreover, the cost and time associated with the manufacture of neoantigen vaccines is significant.

Thus, there remains a need in the art to efficiently and accurately predict effective neoantigen candidates for immunogenic compositions.

### SUMMARY OF THE INVENTION

The disclosure relates generally to methods relating to the detection of mutations, such as genetic fusions or deletions, associated with cancer development that result in expression of a neoantigen. The neoepitope serves as the basis for the manufacture of a vaccine, which can be administered to a subject to induce an immune response against cells producing the neoantigen (e.g., cancer cells).

In one aspect, the disclosure relates to a method for vaccinating a subject against cancer, comprising detecting circulating tumor DNA in a sample from the subject to determine the subject is in need of a cancer vaccine; obtaining nucleic acid sequence information from the subject, wherein the nucleic acid sequence information is used to obtain a peptide sequence of a neoantigen identified by comparing the nucleic acid sequence information from the subject to reference nucleic acid sequence information from normal cells, wherein the peptide sequence of the neoantigen comprises at least one sequence modification that distinguishes it from the corresponding peptide sequence identified from the reference nucleic acid sequence information; and administering the neoantigen to the subject such that the neoantigen is presented to the subject's immune system, thereby vaccinating the subject against cancer.

In certain embodiments, the sequence modification is gene fusion, a deletion, a frameshift mutation, a splice site mutation, a read-through mutation, or an amino acid substitution.

In certain embodiments, the circulating tumor DNA originates from a precancerous cell. In certain embodiments, the circulating tumor DNA originates from a cancerous cell.

In certain embodiments, the circulating tumor DNA is detected using a methylome-based assay. In certain embodiments, the methylome-based assay analyzes methylation of a plurality of CpG sites within a chromosomal region selected from the CpG sites listed in Table 1 of PCT/US2018/032612 and Tables 2 and 3 of PCT/US2021/064210.

In certain embodiments, the neoantigen is derived from a random somatic mutation specific to the subject. In certain embodiments, the neoantigen is derived from a non-random mutation that is shared amongst a population of subjects.

In certain embodiments, the sequence modification is a deletion in chromosome 17p. In certain embodiments, the deletion in chromosome 17p is a deletion in Eif5a and/or Alox15b/Alox8. In certain embodiments, the sequence modification disrupts a TP53 tumor suppressor gene.

In certain embodiments, the nucleic acid sequence information is obtained by long-read sequencing. In certain embodiments, the nucleic acid sequence information is from mRNA. In certain embodiments, the nucleic acid sequence information is obtained by isolating nucleic acid derived from exosomes. In certain embodiments, the method further comprising the step of isolating the exosomes.

In another aspect, the disclosure relates to a method for vaccinating a subject against cancer, wherein the subject has circulating tumor DNA comprising administering a neoantigen to the subject such that the neoantigen is presented to the subject's immune system, thereby vaccinating the subject against cancer; wherein the peptide sequence of the neoantigen is obtained from nucleic acid sequence information from the subject and comprises at least one sequence modification as compared to a corresponding peptide sequence identified from a reference nucleic acid sequence information from normal cells.

In certain embodiments, the sequence modification is gene fusion, a deletion, a frameshift mutation, a splice site mutation, a read-through mutation, or an amino acid substitution.

In certain embodiments, the circulating tumor DNA originates from a precancerous cell. In certain embodiments, the circulating tumor DNA originates from a cancerous cell. In certain embodiments, the circulating tumor DNA was detected using a methylome-based assay. In certain embodiments, the methylome-based assay analyzes methylation of a plurality of CpG sites within a chromosomal region selected from the CpG sites listed in Table 1 of PCT/US2018/032612 and Tables 2 and 3 of PCT/US2021/064210.

In certain embodiments, the neoantigen is derived from a random somatic mutation specific to the subject. In certain embodiments, the neoantigen is derived from a non-random mutation that is shared amongst a population of subjects.

In certain embodiments, the sequence modification is a deletion in chromosome 17p.

In certain embodiments, the deletion in chromosome 17p is a deletion in Eif5a and/or Alox15b/Alox8. In certain embodiments, the sequence modification disrupts a TP53 tumor suppressor gene.

In certain embodiments, the nucleic acid sequence information is obtained by long-read sequencing. In certain embodiments, the nucleic acid sequence information is from mRNA. In certain embodiments, the nucleic acid sequence information is obtained by isolating nucleic acid derived from exosomes. In certain embodiments, the method further comprising the step of isolating the exosomes.

In another aspect, the disclosure relates to a method of producing a peptide or polynucleotide neoantigen cancer vaccine, comprising obtaining nucleic acid sequence information from a subject determined to have circulating tumor DNA, wherein the nucleic acid sequence information is used to obtain a peptide sequence of a neoantigen identified by comparing the nucleic acid sequence information from the subject to reference nucleic acid sequence information from normal cells, wherein the peptide sequence of the neoantigen comprises at least one sequence modification that distinguishes it from the corresponding peptide sequence identified from the reference nucleic acid sequence information; and producing the peptide neoantigen cancer vaccine comprising the peptide sequence of the neoantigen or the polynucleotide neoantigen cancer vaccine encoding the peptide sequence of the neoantigen.

In certain embodiments, the sequence modification is gene fusion, a deletion, a frameshift mutation, a splice site mutation, a read-through mutation, or an amino acid substitution.

In certain embodiments, the circulating tumor DNA originates from a precancerous cell. In certain embodiments, the circulating tumor DNA originates from a cancerous cell.

In certain embodiments, a methylome-based assay was used to determine that the subject had the circulating tumor DNA. In certain embodiments, the methylome-based assay analyzes methylation of a plurality of CpG sites within a chromosomal region selected from the CpG sites listed in Table 1 of PCT/US2018/032612 and Tables 2 and 3 of PCT/US2021/064210.

In certain embodiments, the neoantigen is derived from a random somatic mutation specific to the subject. In certain embodiments, the neoantigen is derived from a non-random mutation that is shared amongst a population of subjects.

In certain embodiments, the sequence modification is a deletion in chromosome 17p. In certain embodiments, the deletion in chromosome 17p is a deletion in Eif5a and/or Alox15b/Alox8. In certain embodiments, the sequence modification disrupts a TP53 tumor suppressor gene.

In certain embodiments, the nucleic acid sequence information is obtained by long-read sequencing. In certain embodiments, the nucleic acid sequence information is from mRNA. In certain embodiments, the nucleic acid sequence information is obtained by isolating nucleic acid derived from exosomes. In certain embodiments, the method further comprising the step of isolating the exosomes.

These and other aspects and features of the invention are described in the following detailed description and claims.

### BRIEF DESCRIPTION OF THE DRAWINGS

The foregoing and other objects, features and advantages of the invention will become apparent from the following description of preferred embodiments, as illustrated in the accompanying drawings. Like referenced elements identify common features in the corresponding drawings. The drawings are not necessarily to scale, with emphasis instead being placed on illustrating the principles of the present invention, in which:

FIG. 1 is a flowchart showing a process for detecting neoantigens for personalized or universal cancer vaccines.

FIG. 2 is a schematic of mRNA vaccine development adapted from Jackson et al., (2020) NPJ VACCINES 5, 11. Non-replicating mRNA (NRM) constructs encode the coding sequence (CDS), and are flanked by 5′ and 3′ untranslated regions (UTRs), a 5′-cap structure and a 3′-poly-(A) tail. The self-amplifying mRNA (SAM) construct encodes additional replicase components able to direct intracellular mRNA amplification. (1) NRM and SAM are formulated in FIG. 2 in lipid nanoparticles (LNPs) that encapsulate the mRNA constructs to protect them from degradation and promote cellular uptake. (2) Cellular uptake of the mRNA with its delivery system typically exploits membrane-derived endocytic pathways. (3) Endosomal escape allows release of the mRNA in to the cytosol. (4) Cytosol-located NRM constructs can also be immediately translated by ribosomes to produce the protein of interest, which undergoes subsequent post-translational modification. (5) SAM constructs can also be immediately translated by ribosomes to produce the replicase machinery when necessary for self-amplification of the mRNA. (6) Self-amplified mRNA constructs are translated by ribosomes to produce the protein of interest, which undergoes subsequent post-translation modification. (7) The expressed proteins of interest are generated as secreted, trans-membrane, or intracellular protein. (8) The innate and adaptive immune responses detect the protein of interest.

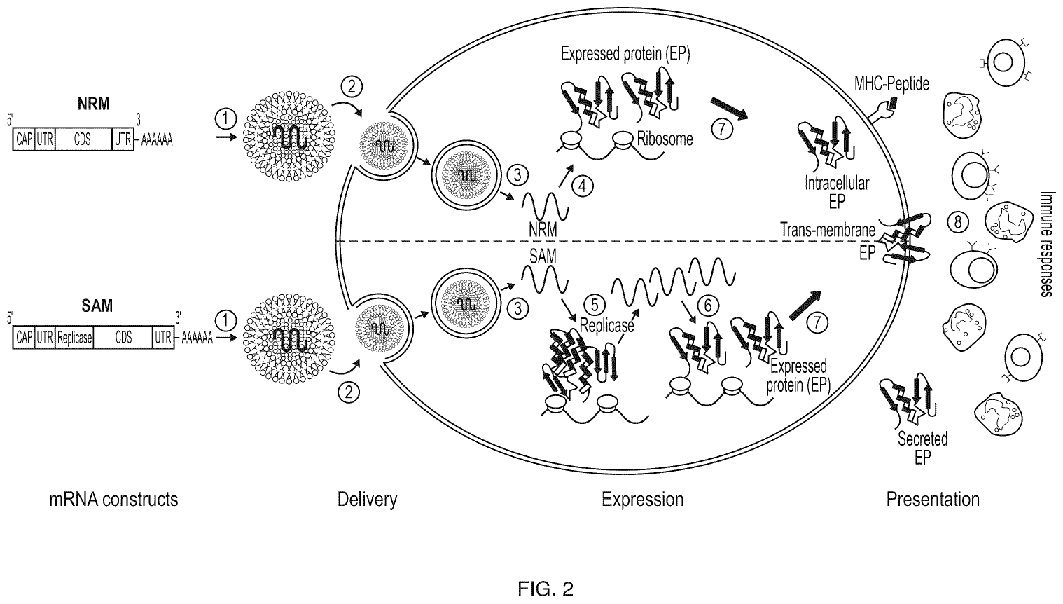

FIG. 3 shows a schematic adapted from Yamagata et al. ((2021) SCI REP 11, 17075) to define a co-extraction method for RNA and DNA; which concluded that using NucleoSpin RNA kit (see Macherey-Nagel. RNA from blood. NucleoSpin® RNA Blood Kit. User Manual. www.mn-net.com/media/pdf/0d/f8/93/Instruction-NucleoSpin-RNA-Blood.pdf) and QIAamp DNA kit (see Qiagen. DNA from blood. QIAamp® DNA Blood Kit. User Manual. file:///Users/cgiley/Downloads/EN-QIAamp-96-DNA-Blood-Handbook.pdf) for extraction yielded the best results. Protocol: 1. Collect blood in EDTA tube and freeze at −20° C. overnight. Transfer to deep freezer at −80° C. until ready for extraction. 2. Thaw the EDTA blood tube in an aluminum block for 18 minutes. 3. Follow the Macherey-Nagel NucleoSpin RNA Blood commercially available kit to extract RNA (see Macherey-Nagel. RNA from blood. NucleoSpin® RNA Blood Kit. User Manual. www.mn-net.com/media/pdf/0d/f8/93/Instruction-NucleoSpin-RNA-Blood.pdf). 4. Follow the Qiagen QIAamp DNA commercially available kit to extract DNA (see Qiagen. DNA from blood. QIAamp® DNA Blood Kit. User Manual. file:///Users/cgiley/Downloads/EN-QIAamp-96-DNA-Blood-Handbook.pdf.

FIG. 4 shows a schematic of a procedure adapted from Qiagen exoRNeasy (see Qiagen. Exosomal RNA from blood. exoRNeasy Midi/Maxi Handbook. www.qiagen.com/us/products/discovery-and-translational-research/exosomes-ctcs/exosomes/exorneasy-midi-and-maxi-kits/). Protocol: 1. Collect blood in EDTA or Streck® collection tube and single-spin to obtain plasma/serum. 2. Transfer plasma/serum and filter large particles (e.g., using Millipore Milex-AA syringe filters). 3. Add XBP buffer to bind exosomes to exoEasy column. 4. Spin at 5000xg for 5 minutes and discard flow-through. 5. Add XWP buffer to wash bound exosomes. 6. Spin at 5000xg for 5 minutes and discard flow-through. 7. Add QIAzol to lyse vesicles. 8. Spin at 5000xg for 5 minutes, retain flow-through and transfer to new tube. 9. Add chloroform and vortex. 10. Incubate for 3 minutes. 11. Spin at 12000xg for 15 minutes at 4° C. 12. Recover clear, aqueous phase and transfer to new tube. 13. Add 100% ethanol and vortex. 14. Add aqueous phase & 100% ethanol solution to RNeasy MinElute column to bind total RNA. 15. Spin at 8000xg for 15 s and discard flow-through. 16. Add RWT buffer to wash bound RNA. 17. Spin at 8000xg for 15 s and discard flow-through. 18. Add RPE buffer to wash bound RNA. 19. Spin at 8000xg for 15 s and discard flow-through. 20. Add RPE buffer to wash bound RNA. 21. Spin at 8000xg for 15 s and discard flow-through. 22. Add RNase-free water to elute bound RNA. 23. Spin at 8000xg for 15 s and retain flow-through of purified exosomal-RNA.

### DETAILED DESCRIPTION

The disclosure relates generally to methods relating to the detection of mutations, such as genetic fusions or deletions, associated with cancer development that results in expression of a neoantigen. The neoepitope serves as the basis for the manufacture of a vaccine, which can be administered to a subject to induce an immune response against cells producing the neoantigen (e.g., cancer cells).

### I. Definitions

As used herein, the term “about” or “approximately” can mean within an acceptable error range for the particular value as determined by one of ordinary skill in the art, which can depend in part on how the value is measured or determined, e.g., the limitations of the measurement system. For example, “about” can mean within 1 or more than 1 standard deviation, per the practice in the art. “About” can mean a range of ±20%, ±10%, ±5%, or ±1% of a given value. The term “about” or “approximately” can mean within an order of magnitude, within 5-fold, or within 2-fold, of a value. Where a particular value is described in the application and claims, unless otherwise stated the term “about” meaning within an acceptable error range for the particular value can be assumed. The term “about” can have the meaning as commonly understood by one of ordinary skill in the art. The term “about” can refer to ±10%. The term “about” can refer to ±5%.

As used herein, the term “biological sample,” or “sample” refers to any sample taken from a subject, which can reflect a biological state associated with the subject, and that includes cell free DNA. A biological sample can take any of a variety of forms, such as a liquid biopsy (e.g., blood, urine, stool, saliva, or mucous), or a tissue biopsy, or other solid biopsy. Examples of biological samples include, but are not limited to, blood, whole blood, plasma, serum, urine, cerebrospinal fluid, fecal, saliva, sweat, tears, pleural fluid, pericardial fluid, or peritoneal fluid of the subject. A biological sample can include any tissue or material derived from a living or dead subject. A biological sample can be a cell-free sample. A biological sample can comprise a nucleic acid (e.g., DNA or RNA) or a fragment thereof. The term “nucleic acid” can refer to deoxyribonucleic acid (DNA), ribonucleic acid (RNA) or any hybrid or fragment thereof. The nucleic acid in the sample can be a cell-free nucleic acid. A sample can be a liquid sample or a solid sample (e.g., a cell or tissue sample). A biological sample can be a bodily fluid, such as blood, plasma, serum, urine, vaginal fluid, fluid from a hydrocele (e.g., of the testis), vaginal flushing fluids, pleural fluid, ascitic fluid, cerebrospinal fluid, saliva, sweat, tears, sputum, bronchoalveolar lavage fluid, discharge fluid from the nipple, aspiration fluid from different parts of the body (e.g., thyroid, breast), etc. A biological sample can be a stool sample. In various embodiments, the majority of DNA in a biological sample that has been enriched for cell-free DNA (e.g., a plasma sample obtained via a centrifugation protocol) can be cell-free (e.g., greater than 50%, 60%, 70%, 80%, 90%, 95%, or 99% of the DNA can be cell-free). A biological sample can be treated to physically disrupt tissue or cell structure (e.g., centrifugation and/or cell lysis), thus releasing intracellular components into a solution which can further contain enzymes, buffers, salts, detergents, and the like which can be used to prepare the sample for analysis.

As used herein, the terms “nucleic acid” and “nucleic acid molecule” are used interchangeably. The terms refer to nucleic acids of any composition form, such as deoxyribonucleic acid (DNA, e.g., complementary DNA (cDNA), genomic DNA (gDNA) and the like), and/or DNA analogs (e.g., containing base analogs, sugar analogs and/or a non-native backbone and the like), all of which can be in single-or double-stranded form. Unless otherwise limited, a nucleic acid can comprise known analogs of natural nucleotides, some of which can function in a similar manner as naturally occurring nucleotides. A nucleic acid can be in any form useful for conducting processes herein (e.g., linear, circular, supercoiled, single-stranded, double-stranded and the like). A nucleic acid in some embodiments can be from a single chromosome or fragment thereof (e.g., a nucleic acid sample may be from one chromosome of a sample obtained from a diploid organism). In certain embodiments nucleic acids comprise nucleosomes, fragments or parts of nucleosomes or nucleosome-like structures. Nucleic acids can comprise protein (e.g., histones, DNA binding proteins, and the like). Nucleic acids analyzed by processes described herein can be substantially isolated and are not substantially associated with protein or other molecules. Nucleic acids can also include derivatives, variants and analogs of DNA synthesized, replicated or amplified from single-stranded (“sense” or “antisense,” “plus” strand or “minus” strand, “forward” reading frame or “reverse” reading frame) and double-stranded polynucleotides. Deoxyribonucleotides can include deoxyadenosine, deoxycytidine, deoxyguanosine and deoxythymidine. A nucleic acid may be prepared using a nucleic acid obtained from a subject as a template.

As used herein, the terms “template nucleic acid” and “template nucleic acid molecule(s)” are used interchangeably. The terms refer to nucleic acid that has been obtained from a sample and processed to form an immortalized library. The template nucleic acid can be nucleic acid obtained directly from the sample, or nucleic acid that is derived from that obtained directly from the sample. Examples of nucleic acid derived from a sample include DNA that has been reverse-transcribed from RNA obtained directly from a sample, or DNA that has be amplified from DNA obtained directly from a sample, for example, by PCR.

As used herein, the term “cell-free nucleic acids” refers to nucleic acid molecules that can be found outside cells, in bodily fluids such as blood, whole blood, plasma, serum, urine, cerebrospinal fluid, fecal, saliva, sweat, sweat, tears, pleural fluid, pericardial fluid, or peritoneal fluid of a subject. Cell-free nucleic acids originate from one or more healthy cells and/or from one or more cancer cells, or from non-human sources such bacteria, fungi, viruses. Examples of the cell-free nucleic acids include but are not limited to cell-free DNA (“cfDNA”), including mitochondrial DNA or genomic DNA, and cell-free RNA. In certain embodiments herein, the cfDNA is circulating tumor DNA. In certain embodiments herein, instruments for assessing the quality of the cell-free nucleic acids, such as the TapeStation System from Agilent Technologies (Santa Clara, CA) can be used. Concentrating low-abundance cfDNA can be accomplished, for example using a Qubit Fluorometer from Thermofisher Scientific (Waltham, MA).

As used herein, the term “methylation” refers to a modification of a nucleic acid where a hydrogen atom on the pyrimidine ring of a cytosine base is converted to a methyl group, forming 5-methylcytosine. Methylation can occur at dinucleotides of cytosine and guanine referred to herein as “CpG sites”. Methylation of cytosine can occur in cytosines in other sequence contexts, for example, 5′-CHG-3′ and 5′-CHH-3′, where His adenine, cytosine or thymine. Cytosine methylation can also be in the form of 5-hydroxymethylcytosine. Methylation of DNA can include methylation of non-cytosine nucleotides, such as N6-methyladenine. Anomalous cfDNA methylation can be identified as hypermethylation or hypomethylation, both of which may be indicative of cancer status. As is well known in the art, DNA methylation anomalies (compared to healthy controls) can cause different effects, which may contribute to cancer.

As used herein the term “methylation index” for each genomic site (e.g., a CpG site, a region of DNA where a cytosine nucleotide is followed by a guanine nucleotide in the linear sequence of bases along its 5′→3′ direction) can refer to the proportion of sequence reads showing methylation at the site over the total number of reads covering that site. The “methylation density” of a region can be the number of reads at sites within a region showing methylation divided by the total number of reads covering the sites in the region. The sites can have specific characteristics, (e.g., the sites can be CpG sites). The “CpG methylation density” of a region can be the number of reads showing CpG methylation divided by the total number of reads covering CpG sites in the region (e.g., a particular CpG site, CpG sites within a CpG island, or a larger region). For example, the methylation density for each 100-kb bin in the human genome can be determined from the total number of unconverted cytosines (which can correspond to methylated cytosine) at CpG sites as a proportion of all CpG sites covered by sequence reads mapped to the 100-kb region. In some embodiments, this analysis is performed for other bin sizes, e.g., 50-kb or 1-Mb, etc. In some embodiments, a region is an entire genome or a chromosome or part of a chromosome (e.g., a chromosomal arm). A methylation index of a CpG site can be the same as the methylation density for a region when the region includes that CpG site. The “proportion of methylated cytosines” can refer the number of cytosine sites, “C's,” that are shown to be methylated (for example unconverted after bisulfite conversion) over the total number of analyzed cytosine residues, e.g., including cytosines outside of the CpG context, in the region. The methylation index, methylation density and proportion of methylated cytosines are examples of “methylation levels.”

In various embodiments, performing deamination of cytosine residues is useful for determining methylation statuses of nucleic acids from a sample. Performing deamination involves providing or exposing nucleic acids from a sample to a deaminating agent. In various embodiments, performing deamination of cytosine residues involves performing selective deamination. Selective deamination refers to a process in which cytosine residues are selectively deaminated over 5-methylcytosine residues. Deamination of cytosine forms uracil, effectively inducing a C to T point mutation to allow for detection of methylated cytosines. Methods of deaminating cytosine are known in the art, and include bisulfite conversion and enzymatic conversion. Bisulfite conversion enables highly efficient conversion of unmethylated cytosines to uracils of DNA from samples such as whole blood or plasma, cultured cells, tissue samples, genomic DNA, and formalin-fixed, paraffin-embedded (FFPE) tissues. Bisulfite conversion can be performed using commercially available technologies, such as Zymo Gold available from Zymo Research (Irvine, CA) or EpiTect Fast available from Qiagen (Germantown, MD). In certain embodiments, the enzymatic conversion comprises subjecting the nucleic acid to TET2, which oxidizes methylated cytosines, thereby protecting them, and subsequent exposure to APOBEC, which converts unprotected (unmethylated) cytosines to uracils.

Certain portions of a genome comprise regions with a high frequency of CpG sites. A CpG site is portion of a genome that has cytosine and guanine separated by only one phosphate group and is often denoted as “5′—C-phosphate—G—3′”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIS and features of the genome are referred to herein as “cancer informative CGIs”. An “informative CpG” can be specified by reference to a specific CpG site, or to a collection of one or more CpG sites by reference to a CG island that contains the collection. These cancer informative CGIs tend to have methylation patterns in tumor cells that are different from the methylation patterns in healthy cells. DNA fragments from other CGIs may not express such differences.

As used herein, the term “methylation profile” (also called methylation status) can include information related to DNA methylation for a region. Information related to DNA methylation can include a methylation index of a CpG site, a methylation density of CpG sites in a region, a distribution of CpG sites over a contiguous region, a pattern or level of methylation for each individual CpG site within a region that contains more than one CpG site, and non-CpG methylation. A methylation profile of a substantial part of the genome can be considered equivalent to the methylome. “DNA methylation” in mammalian genomes can refer to the addition of a methyl group to position 5 of the heterocyclic ring of cytosine (e.g., to produce 5-methylcytosine) among CpG dinucleotides. Methylation of cytosine can occur in cytosines in other sequence contexts, for example, 5′-CHG-3′ and 5′-CHG-3′ where His adenine, cytosine or thymine. Cytosine methylation can also be in the form of 5-hydroxymethylcytosine. Methylation of DNA can include methylation of non-cytosine nucleotides, such as N6-methyladenine.

As used herein, the term “methylome-based assay” refers to an assay that detects a methylation profile in a sample from a subject. Non-limiting examples of methylome-based assays include mass spectrometry, methylation-specific PCR, whole genome bisulfite sequencing (BS-Seq), reduced representation bisulfite sequencing (RRBS), the HELP assay, GLAD-PCR assay, ChIP-on-chip assay, restriction landmark genomic scanning, methylated DNA immunoprecipitation (MeDIP), methylation specific bisulfite sequencing (MSBS), pyrosequencing of bisulfite-treated DNA, molecular break light assay for DNA adenine methyltransferase activity, Methyl Sensitive Southern Blotting, high resolution melt analysis (HRM or HRMA), methylation sensitive single nucleotide primer extension assay (msSNuPE), Illumina Methylation Assay, and nanopore sequencing.

As used herein, the term “long-read sequencing” refers to methods of sequencing a nucleic acid that include an average read length that is longer than standard sequencing methods. As described herein, long-read sequencing refers to methods of sequencing that may including an average read length that is, for example, greater than 500 bases in length. Generally, long sequence reads include an average read length that is longer than sequence reads obtained through standard sequencing methods. In various embodiments, the long sequence reads refer to sequence reads of at least 500 bases, at least 1 kilobase, at least 2 kilobases (kb), at least 3 kb, at least 4 kb, at least 5 kb, at least 6 kb, at least 7 kb, at least 8 kb, at least 9 kb, at least 10 kb, at least 12 kb, at least 15 kb, at least 20 kb, at least 25 kb, at least 30 kb, at least 40 kb, at least 50 kb, at least 60 kb, at least 70 kb, at least 80 kb, at least 90 kb, at least 100 kb, at least 200 kb, at least 300 kb, at least 400 kb, at least 500 kb, at least 600 kb, at least 700 kb, at least 800 kb, at least 900 kb, at least 1000 kb, at least 1500 kb, or at least 2000 kb. In particular embodiments, long sequence reads refer to sequence reads of between 5 kb and 100 kb, between 10 kb and 80 kb, between 20 kb and 70 kb, between 30 kb and 60 kb, or between 40 kb and 50 kb. In particular embodiments, long sequence reads are greater than about 8 kb, greater than about 9 kb or greater than about 10 kb. In particular embodiments, long sequence reads are between about 10 kb and about 100 kb, or between about 10 kb and about 2 MB. Methods for long-read sequencing are known in the art and such methods can be performed using, for example, an Oxford Nanopore instrument (e.g., PromethION™) or Pacific Biosciences Single-Molecule Real-Time (SMRT) sequencing technology.

As used herein, the term “amplifying” means performing an amplification reaction. In one aspect, an amplification reaction is “template-driven” in that base pairing of reactants, either nucleotides or oligonucleotides, have complements in a template polynucleotide that are required for the creation of reaction products. In one aspect, template-driven reactions are primer extensions with a nucleic acid polymerase, or oligonucleotide ligations with a nucleic acid ligase. Such reactions include, but are not limited to, polymerase chain reactions (PCRs), linear polymerase reactions, nucleic acid sequence-based amplification (NASBAs), rolling circle amplifications, and the like, disclosed in the following references, each of which are incorporated herein by reference herein in their entirety: Mullis et al., U.S. Pat. Nos. 4,683,195; 4,965,188; 4,683,202; 4,800,159 (PCR); Gelfand et al., U.S. Pat. No. 5,210,015 (real-time PCR with “taqman” probes); Wittwer et al., U.S. Pat. No. 6,174,670; Kacian et al., U.S. Pat. No. 5,399,491 (“NASBA”); Lizardi, U.S. Pat. No. 5,854,033; Aono et al., Japanese patent publ. JP 4-262799 (rolling circle amplification); and the like. In one aspect, the amplification reaction is PCR. An amplification reaction may be a “real-time” amplification if a detection chemistry is available that permits a reaction product to be measured as the amplification reaction progresses, e.g., “real-time PCR”, or “real-time NASBA” as described in Leone et al., Nucleic Acids Research, 26:2150-2155 (1998), and like references.

A “reaction mixture” means a solution containing all the necessary reactants for performing a reaction, which may include, but is not be limited to, buffering agents to maintain pH at a selected level during a reaction, salts, co-factors, scavengers, and the like.

The terms “fragment” or “segment”, as used interchangeably herein, refer to a portion of a larger polynucleotide molecule. A polynucleotide, for example, can be broken up, or fragmented into, a plurality of segments. Various methods of fragmenting nucleic acid are well known in the art. These methods may be, for example, either chemical or physical or enzymatic in nature. Enzymatic fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave a polynucleotide at known or unknown locations. Physical fragmentation methods may involve subjecting a polynucleotide to a high shear rate. High shear rates may be produced, for example, by moving DNA through a chamber or channel with pits or spikes, or forcing a DNA sample through a restricted size flow passage, e.g., an aperture having a cross sectional dimension in the micron or submicron range. Other physical methods include sonication and nebulization. Combinations of physical and chemical fragmentation methods may likewise be employed, such as fragmentation by heat and ion-mediated hydrolysis. See, e.g., Sambrook et al., “Molecular Cloning: A Laboratory Manual,” 3rd Ed. Cold Spring Harbor Laboratory Press, Cold Spring Harbor, N.Y. (2001) which is incorporated herein by reference for all purposes. These methods can be optimized to digest a nucleic acid into fragments of a selected size range.

The terms “polymerase chain reaction” or “PCR”, as used interchangeably herein, mean a reaction for the in vitro amplification of specific DNA sequences by the simultaneous primer extension of complementary strands of DNA. In other words, PCR is a reaction for making multiple copies or replicates of a target nucleic acid flanked by primer binding sites, such reaction comprising one or more repetitions of the following steps: (i) denaturing the target nucleic acid, (ii) annealing primers to the primer binding sites, and (iii) extending the primers by a nucleic acid polymerase in the presence of nucleoside triphosphates. Usually, the reaction is cycled through different temperatures optimized for each step in a thermal cycler instrument. Particular temperatures, durations at each step, and rates of change between steps depend on many factors that are well-known to those of ordinary skill in the art, e.g., exemplified by the following references: McPherson et al., editors, PCR: A Practical Approach and PCR2: A Practical Approach (IRL Press, Oxford, 1991 and 1995, respectively). For example, in a conventional PCR using Taq DNA polymerase, a double stranded target nucleic acid may be denatured at a temperature>90° C., primers annealed at a temperature in the range 50-75° C., and primers extended at a temperature in the range 72-78° C. The term “PCR” encompasses derivative forms of the reaction, including, but not limited to, RT-PCR, real-time PCR, nested PCR, quantitative PCR, multiplexed PCR, and the like. The particular format of PCR being employed is discernible by one skilled in the art from the context of an application. Reaction volumes can range from a few hundred nanoliters, e.g., 200 nL, to a few hundred μL, e.g., 200 μL. “Reverse transcription PCR,” or “RT-PCR,” means a PCR that is preceded by a reverse transcription reaction that converts a target RNA to a complementary single stranded DNA, which is then amplified, an example of which is described in Tecott et al., U.S. Pat. No. 5,168,038, the disclosure of which is incorporated herein by reference in its entirety. “Real-time PCR” means a PCR for which the amount of reaction product, i.e., amplicon, is monitored as the reaction proceeds. There are many forms of real-time PCR that differ mainly in the detection chemistries used for monitoring the reaction product, e.g., Gelfand et al., U.S. Pat. No. 5,210,015 (“taqman”); Wittwer et al., U.S. Pat. Nos. 6,174,670 and 6,569,627 (intercalating dyes); Tyagi et al., U.S. Pat. No. 5,925,517 (molecular beacons); the disclosures of which are hereby incorporated by reference herein in their entireties. Detection chemistries for real-time PCR are reviewed in Mackay et al., NUCLEIC ACIDS RESEARCH, 30: 1292-1305 (2002), which is also incorporated herein by reference. “Nested PCR” means a two-stage PCR wherein the amplicon of a first PCR becomes the sample for a second PCR using a new set of primers, at least one of which binds to an interior location of the first amplicon. As used herein, “initial primers” in reference to a nested amplification reaction mean the primers used to generate a first amplicon, and “secondary primers” mean the one or more primers used to generate a second, or nested, amplicon. “Asymmetric PCR” means a PCR wherein one of the two primers employed is in great excess concentration so that the reaction is primarily a linear amplification in which one of the two strands of a target nucleic acid is preferentially copied. The excess concentration of asymmetric PCR primers may be expressed as a concentration ratio. Typical ratios are in the range of from 10 to 100. “Multiplexed PCR” means a PCR wherein multiple target sequences (or a single target sequence and one or more reference sequences) are simultaneously carried out in the same reaction mixture, e.g., Bernard et al., ANAL. BIOCHEM., 273: 221-228 (1999) (two-color real-time PCR). Usually, distinct sets of primers are employed for each sequence being amplified. Typically, the number of target sequences in a multiplex PCR is in the range of from 2 to 50, or from 2 to 40, or from 2 to 30. “Quantitative PCR” means a PCR designed to measure the abundance of one or more specific target sequences in a sample or specimen. Quantitative PCR includes both absolute quantitation and relative quantitation of such target sequences. Quantitative measurements are made using one or more reference sequences or internal standards that may be assayed separately or together with a target sequence. The reference sequence may be endogenous or exogenous to a sample or specimen, and in the latter case, may comprise one or more competitor templates. Typical endogenous reference sequences include segments of transcripts of the following genes: β-actin, GAPDH, β2-microglobulin, ribosomal RNA, and the like. Techniques for quantitative PCR are well-known to those of ordinary skill in the art, as exemplified in the following references, which are incorporated by reference herein in their entireties: Freeman et al., BIOTECHNIQUES, 26: 112-126 (1999); Becker-Andre et al., NUCLEIC ACIDS RESEARCH, 17: 9437-9447 (1989); Zimmerman et al., BIOTECHNIQUES, 21: 268-279 (1996); Diviacco et al., GENE, 122: 3013-3020 (1992); and Becker-Andre et al., NUCLEIC ACIDS RESEARCH, 17: 9437-9446 (1989).

The term “primer” as used herein means an oligonucleotide, either natural or synthetic, that is capable, upon forming a duplex with a polynucleotide template, of acting as a point of initiation of nucleic acid synthesis and being extended from its 3′ end along the template so that an extended duplex is formed. Extension of a primer is usually carried out with a nucleic acid polymerase, such as a DNA or RNA polymerase. The sequence of nucleotides added in the extension process is determined by the sequence of the template polynucleotide. Usually, primers are extended by a DNA polymerase. Primers usually have a length in the range of from 14 to 40 nucleotides, or in the range of from 18 to 36 nucleotides. Primers are employed in a variety of nucleic amplification reactions, for example, linear amplification reactions using a single primer, or polymerase chain reactions, employing two or more primers. Guidance for selecting the lengths and sequences of primers for particular applications is well known to those of ordinary skill in the art, as evidenced by the following reference that is incorporated by reference herein in its entirety: Dieffenbach, editor, PCR Primer: A Laboratory Manual, 2nd Edition (Cold Spring Harbor Press, New York, 2003).

As used herein, the term “sensitivity” refers to the ability of a diagnostic assay to correctly identify subjects with a condition of interest. As used herein, the term “specificity” refers to the ability of a diagnostic assay to correctly identify subjects without a condition of interest.

As used herein, the term “subject” and “patient” are used interchangeably herein and refer to any living or non-living organism, including but not limited to a human (e.g., a male human, female human, fetus, pregnant female, child, or the like), a non-human animal, a plant, a bacterium, a fungus or a protist. Any human or non-human animal can serve as a subject, including but not limited to mammal, reptile, avian, amphibian, fish, ungulate, ruminant, bovine (e.g., cattle), equine (e.g., horse), caprine and ovine (e.g., sheep, goat), swine (e.g., pig), camelid (e.g., camel, llama, alpaca), monkey, ape (e.g., gorilla, chimpanzee), ursid (e.g., bear), poultry, dog, cat, mouse, rat, fish, dolphin, whale and shark. In some embodiments, a subject is a male or female of any age (e.g., a man, a women or a child).

### II. Methods of Detecting Circulating Tumor DNA

Circulating tumor DNA refers to DNA present in the bloodstream of a subject that originated from a tumor cell (or a “cancer cell”, which is used interchangeably herein) or a precancerous cell. For example, tumor cells that are apoptotic or necrotic release tumor DNA into the bloodstream, which can be detected in a sample of cell free DNA (cfDNA), indicating the presence of a tumor in the subject.

The particular method used to detect circulating tumor DNA may depend on whether there is prior knowledge of a biomarker (e.g., a mutation or methylation pattern) specific to a subject's tumor, e.g., as a result of biomarker analysis of a tumor sample (e.g., a biopsy) from a subject. In another approach, where there is no prior knowledge of biomarkers in the original tumor, a broad panel of the most frequently mutated genes (e.g., the Catalogue of Somatic Mutations in Cancer (COSMIC)) or of cancer-informative CGIs can be used to assess the presence of circulating tumor DNA. In certain embodiments herein, circulating tumor DNA can be detected using a methylome-based assay, e.g., to detect cancer-informative CGIs.

In certain embodiments, the methylome-based assay involves analyzing methylation statuses of a plurality of CGIs. Cancer informative CGI can be a “CGI identifier” or reference number to allow referencing CGIs during data processing by their respective unique CGI identifiers. The accompanying tables (e.g., Tables 1-4) lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4.

A CpG site is portion of a genome that has cytosine and guanine separated by only one phosphate group and is often denoted as “5′—C-phosphate—G—3′”, or “CpG” for short. Regions with a high frequency of CpG sites are commonly referred to as “CG islands” or “CGIs”. It has been found that certain CGIs and certain features of certain CGIs in tumor cells tend to be different from the same CGIs or features of the CGIs in healthy cells. Herein, such CGIs and features of the genome are referred to herein as “cancer informative CGIs.” Cancer informative CGI can be a “CGI identifier” or reference number to allow referencing CGIs during data processing by their respective unique CGI identifiers. Example CGIs include, but are not limited to, the CGIs shown in the accompanying tables (referred to herein as Tables 1-4) which lists, for each CGI, its respective location in the human genome. Additional example CGIs are disclosed in WO2018209361 (see Table 1) and WO2022133315 (see Table 2 entitled “TOO Methylation Sites” and Table 3 entitled “Pan Cancer Methylation Sites”), each of which is hereby incorporated by reference in its entirety. In some embodiments, methylation statuses of a plurality of CpGs within a CGI may be analyzed. In some embodiments, at least a portion of the CpGs within a CGI may be analyzed. In other embodiments, all of the CpGs within a CGI may be analyzed. In some embodiments, an analysis of a CGI as contemplated herein may comprise analyzing CpGs within at least a portion of one or more regions in Tables 1-4 of the instant specification.

In certain embodiments, the methylome-based assay involves analyzing methylation statuses of a plurality of CGIs. In various embodiments, the methylome-based assay involves analyzing all of the CGIs in any one of Tables 1, 2, 3, or 4. In various embodiments, the methylome-based assay involves analyzing at most 10% of the CGIs in Table 1. In various embodiments, the methylome-based assay involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 1. In various embodiments, the methylome-based assay involves analyzing at most 10% of the CGIs in Table 2. In various embodiments, the methylome-based assay involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 2. In various embodiments, the methylome-based assay involves analyzing at most 10% of the CGIs in Table 3. In various embodiments, the methylome-based assay involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 3. In various embodiments, the methylome-based assay involves analyzing at most 10% of the CGIs in Table 4. In various embodiments, the methylome-based assay involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Table 4. In various embodiments, the methylome-based assay involves analyzing at most 10% of the CGIs in Tables 2 and 3. In various embodiments, the methylome-based assay involves analyzing at most 10%, at most 20%, at most 30%, at most 40%, at most 50%, at most 55%, at most 60%, at most 65%, at most 70%, at most 75%, at most 80%, at most 85%, at most 90%, at most 91%, at most 92%, at most 93%, at most 94%, at most 95%, at most 96%, at most 97%, at most 98%, or at most 99% of the CGIs in Tables 2 and 3.

In various embodiments, the methylome-based assay involves analyzing 1 CGI, 2 CGIs, 3 CGIs, 4 CGIs, 5 CGIs, 6 CGIs, 7 CGIs, 8 CGIs, 9 CGIs, 10 CGIs, 11 CGIs, 12 CGIs, 13 CGIs, 14 CGIs, 15 CGIs, 16 CGIs, 17 CGIs, 18 CGIs, 19 CGIs, 20 CGIs, 21 CGIs, 22 CGIs, 23 CGIs, 24 CGIs, 25 CGIs, 26 CGIs, 27 CGIs, 28 CGIs, 29 CGIs, 30 CGIs, 31 CGIs, 32 CGIs, 33 CGIs, 34 CGIs, 35 CGIs, 36 CGIs, 37 CGIs, 38 CGIs, 39 CGIs, 40 CGIs, 41 CGIs, 42 CGIs, 43 CGIs, 44 CGIs, 45 CGIs, 46 CGIs, 47 CGIs, 48 CGIs, 49 CGIs, or 50 CGIs (e.g., CGIs as shown in any of Tables 1-4 or portions of CGIs shown in any of Tables 1-4). In various embodiments, the methylome-based assay involves analyzing at most 2 CGIs, at most 5 CGIs, at most 10 CGIs, at most 15 CGIs, at most 20 CGIs, at most 25 CGIs, at most 30 CGIs, at most 35 CGIs, at most 40 CGIs, at most 45 CGIs, or at most 50 CGIs (e.g., CGIs as shown in any of Tables 1-4 or portions of CGIs shown in any of Tables 1-4). In various embodiments, the methylome-based assay involves analyzing at most 50 CGIs, at most 100 CGIs, at most 150 CGIs, at most 200 CGIs, at most 300 CGIs, at most 400 CGIs, at most 500 CGIs, at most 600 CGIs, at most 700 CGIs, at most 800 CGIs, at most 900 CGIs, at most 1000 CGIs, at most 1500 CGIs, at most 2000 CGIs, at most 2500 CGIs, at most 3000 CGIs, at most 3500 CGIs, at most 4000 CGIs, at most 4500 CGIs, at most 5000 CGIs, at most 5500 CGIs, or at most 6000 CGIs (e.g., CGIs as shown in any of Tables 1-4 or portions of CGIs shown in any of Tables 1-4). In particular embodiments, the methylome-based assay involves analyzing at most 500 CGIs.

In certain embodiments, circulating tumor DNA is detected in a single part test (a single phase test). In certain embodiments, a test for circulating tumor DNA is detected in a multi-phase test, such as a two phase test. A first phase (phase 1, screening) can include a non-invasive assay, preferably a rapid assay that uses small amounts of a target specimen (e.g., DNA, RNA, protein, etc.) from a sample. In certain embodiments, phase 1 testing can be lower specificity (e.g., about 95% specificity, 5% false positives; for example, 91% to 99% specificity, 92% to 98% specificity, 93% to 97% specificity, 94% to 96% specificity) but higher sensitivity (e.g., about 80% sensitivity, 20% false negatives; for example, 75% to 85% sensitivity, 76% to 84% sensitivity, 77% to 83% sensitivity, 78 to 82% sensitivity, 79% to 81% sensitivity) in order to screen a large proportion of the testing population rapidly and inexpensively. Samples that are phase 1 (screening) positive will move to a second phase (phase 2, confirmatory). Phase 1 detection can be designed to remove 90-95% of non-cancer patient samples from moving forward for further testing. In certain embodiments, phase 1 testing produces a quantitative result. In certain embodiments, phase 1 testing produces a qualitative result. In certain embodiments, phase 1 testing produces a quantitative and qualitative result.

A second phase (phase 2, confirmatory) can include a more complex assay, which may use larger amounts of the target specimen (e.g., DNA, RNA, protein, etc.). Phase 2 testing is typically higher specificity (e.g., about 90% specificity, 10% false positives; for example, 85% to 95% specificity, 86% to 94% specificity, 87% to 93% specificity, 88% to 92% specificity, 89% to 91% specificity) but lower sensitivity (e.g., about 90% sensitivity, 10% false negatives; for example, 85% to 95% sensitivity, 86% to 94% sensitivity, 87% to 93% sensitivity, 88% to 92% sensitivity, 89% to 91% sensitivity) in order to limit false positives. In certain embodiments, phase 2 testing produces a quantitative result. In certain embodiments, phase 2 testing produces a qualitative result. In certain embodiments, phase 2 testing produces a quantitative and qualitative result.

In various embodiments, the test (e.g., single part test or any phase of a multi-part test, such as phase 1 or phase 2 a two phase test) achieves a certain sensitivity, such as at least 60% sensitivity, in detecting presence of a circulating tumor DNA. In various embodiments, the test achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% sensitivity. In particular embodiments, the test achieves at least 75% sensitivity. In particular embodiments, the test achieves at least 76% sensitivity. In particular embodiments, the test achieves at least 77% sensitivity. In particular embodiments, the test achieves at least 78% sensitivity. In particular embodiments, the test achieves at least 79% sensitivity. In particular embodiments, the test achieves at least 80% sensitivity.

In various embodiments, the test (e.g., single part test or any phase of a multi-part test, such as phase 1 or phase 2 a two phase test) achieves a certain specificity, such as at least 60% specificity, in excluding individuals without circulating tumor DNA. In various embodiments, the test achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% specificity. In particular embodiments, the test achieves at least 90% specificity. In particular embodiments, the test achieves at least 91% specificity. In particular embodiments, the test achieves at least 92% specificity. In particular embodiments, the test achieves at least 93% specificity. In particular embodiments, the test achieves at least 94% specificity. In particular embodiments, the test achieves at least 95% specificity.

In various embodiments, the test achieves at least 15% positive predictive value. In various embodiments, the test achieves at least 15%, at least 16%, at least 17%, at least 18%, at least 19%, at least 20%, at least 21%, at least 22%, at least 23%, at least 24%, at least 25%, at least 26%, at least 27%, at least 28%, at least 29%, at least 30%, at least 31%, at least 32%, at least 33%, at least 34%, at least 35%, at least 36%, at least 37%, at least 38%, at least 39%, or at least 40% positive predictive value. In particular embodiments, the test achieves at least 20% positive predictive value. In particular embodiments, the test achieves at least 21% positive predictive value. In particular embodiments, the test achieves at least 22% positive predictive value. In particular embodiments, the test achieves at least 23% positive predictive value. In particular embodiments, the test achieves at least 24% positive predictive value. In particular embodiments, the test achieves at least 25% positive predictive value. In particular embodiments, the test achieves at least 26% positive predictive value. In particular embodiments, the test achieves at least 27% positive predictive value. In particular embodiments, the test achieves at least 28% positive predictive value. In particular embodiments, the test achieves at least 29% positive predictive value. In particular embodiments, the test achieves at least 30% positive predictive value. In particular embodiments, the test achieves at least 31% positive predictive value. In particular embodiments, the test achieves at least 32% positive predictive value. In particular embodiments, the test achieves at least 33% positive predictive value. In particular embodiments, the test achieves at least 34% positive predictive value. In particular embodiments, the test achieves at least 35% positive predictive value. In particular embodiments, the test achieves at least 36% positive predictive value. In particular embodiments, the test achieves at least 37% positive predictive value. In particular embodiments, the test achieves at least 38% positive predictive value. In particular embodiments, the test achieves at least 39% positive predictive value. In particular embodiments, the test achieves at least 40% positive predictive value.

In various embodiments, the test achieves at least 60% negative predictive value. In various embodiments, the test achieves at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, at least 99.1%, at least 99.2%, at least 99.3%, at least 99.4%, at least 99.5%, at least 99.6%, at least 99.7%, at least 99.8%, or at least 99.9% negative predictive value. In particular embodiments, the test achieves at least 95% negative predictive value. In particular embodiments, the test achieves at least 96% negative predictive value. In particular embodiments, the test achieves at least 97% negative predictive value. In particular embodiments, the test achieves at least 98% negative predictive value. In particular embodiments, the test achieves at least 99% negative predictive value.

### A. Preparing Target Specimen

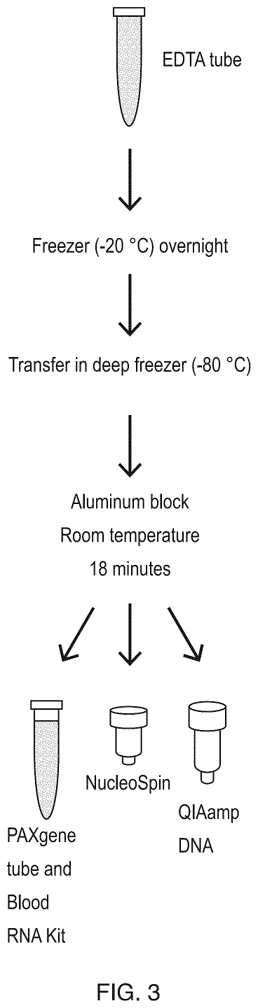

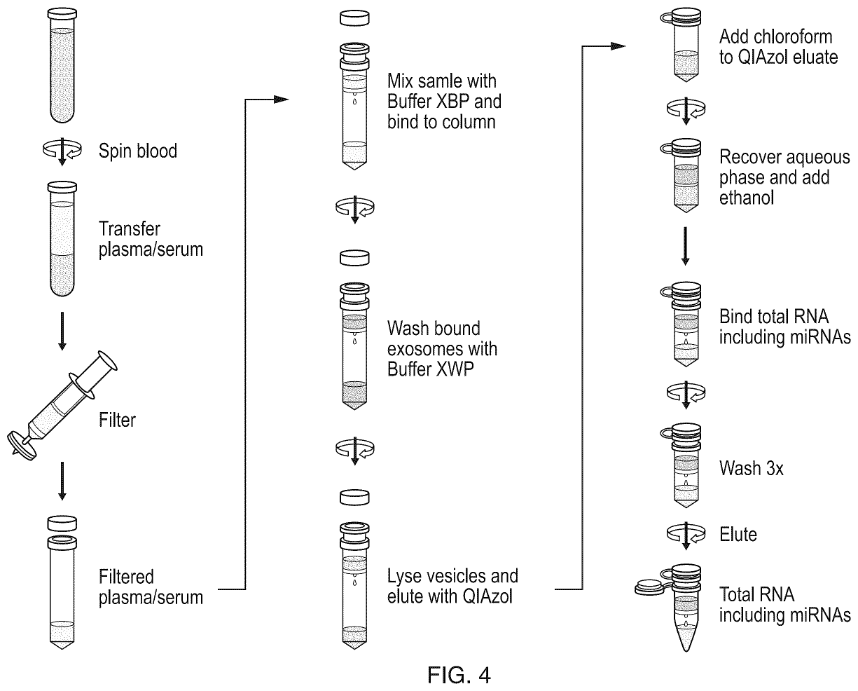

The target specimen type (e.g., DNA, RNA, protein, exosomes, metabolites, etc.) is isolated from a subject's sample (e.g., tissue, blood, plasma, serum, saliva, feces, etc.). See, for example, protocols in FIGS. 3 and 4. Target specimens may be assayed for quality and quantity measurements.

### B. Exemplary Phase 1 Testing (Screening)

A first phase (phase 1, screening) includes a non-invasive assay, preferably a rapid assay that uses small amounts of a target specimen (e.g., DNA, RNA, protein, etc.) from a sample. As discussed above, in certain embodiments, phase 1 testing can be lower specificity (e.g., 95% specificity, 5% false positives) but higher sensitivity (e.g., 80% sensitivity, 20% false negatives) in order to screen a large proportion of the testing population rapidly and inexpensively.

Exemplary phase 1 assays include, but are not limited to, Bradford protein assays, ELISA assays, PCR assays, real-time PCR assays, quantitative real-time PCR (qPCR) assays, allele-specific PCR assays, reverse-transcription PCR assays and reporter assays.

An exemplary protocol of an allele-specific real-time PCR assay is as follows:
1. This assay runs all DNA samples in triplicate with 2 ng input in 5 μL for the reference and mutation assays.
2. Combine 900 nmol/L unspecific primer(s), 100 nmol/L target probe(s), 2X polymerase enzyme(s), 2X dNTPs, 2X passive reference dyes, 10 uL water and 2 ng sample DNA at a pre-specified reaction volume as the reference control assay.
3. Combine 450 nmol/L allele-specific primer(s), 100 nmol/L target probe(s), 2X polymerase enzyme(s), 2X dNTPs, 2X passive reference dyes, 10 uL water and 2 ng sample DNA at a pre-specified reaction volume as the mutation assay.
4. Mix each reaction 10X and centrifuge to collect volume at the bottom of the well or tube.
5. Run the real-time PCR on a calibrated Real-Time PCR system under the following conditions: (1) 95° C. for 10 minutes followed by (2) 50 cycles of 90° C. for 15 seconds and 60° C. for 1 minute with fluorescence detection using FAM/VIC fluorophores.
6. Cycle threshold (Ct) values are recorded by the system and exported into an analysis program (e.g. Excel).
7. Average the Ct values between sample replicates for the reference and mutation assays.
8. Calculate the DCt between the sample average allele-specific Ct minus the sample average unspecific (reference) Ct.
9. Positive mutation results are identified by the DCt cut off >3 cycles and will move forward to phase 2 testing.

Further details of such assays are available at, for example, Lang et al. (2011) J. MOL. DIAG: 13 (1), 23-28; Mitchell et al. (2008) PNAS: 105 (30):10513-10518; Chubarov et al. (2020) DIAGNOSTICS: 10, 872-886; Fox et al. (1998) BRITISH J. CANCER: 77(8),1267-1274; Wong et al. (2005) BIOTECHNIQUES 39:1, 75-85).

Allele-specific real-time PCR can be performed by combining library DNA with PCR reagents and primers specific for target sequences (see Lang et al. (2011), supra). The primers are designed to have single-base discrimination between tumor and non-tumor sequences. Real-time PCR (or digital PCR) can be performed for 30-50 cycles and the output monitored for signal via fluorescence from amplified target DNA or probe sequence. Cycle threshold values (Ct) can be recorded and exported for analysis. The delta-Ct between negative control, positive control, and sample can be calculated to determine presence or absence of target tumor sequences (see Mitchell et al. (2008) supra; Chubarov et al. (2020) supra; Fox et al. (1998) supra; Wong et al. (2005) supra). Slight modifications of this protocol can allow for end-point PCR detection of RNA or DNA of tumor sequences.

ELISA assay detection of target molecules can be performed by coating an immunoassay well with monoclonal antibody designed to specifically detect target molecules (see Corey et al. (1997) INT. J. CANCER 71,1019-1028), followed by blocking against non-specific binding. Target sample is introduced to the well, incubated and washed away. Any bound target can then be bound by a polyclonal antibody specific for the target. Additional secondary antibodies with color or fluorescent tags can be used to detect the presence of target molecules.

### C. Exemplary Phase 2 (Confirmatory) Testing

Phase 2 testing can be a more complex assay, which may use larger amounts of the target specimen (e.g., DNA, RNA, protein, etc.). The result of this assay may be both qualitative and quantitative. As discussed above, phase 2 testing is typically higher specificity (e.g., 90% specificity, 10% false positives) but lower sensitivity (e.g., 90% sensitivity, 10% false negatives) in order to limit false positives.

Exemplary phase 2 assays include, but are not limited to, Next Generation Sequencing (NGS) assays utilizing target enrichment technologies, targeted amplicon sequencing technologies, and/or whole genome sequencing.

In certain embodiment, the phase 2 assay includes construction of a sequence library to sequence cell-free DNA from a subject's sample. The library can be constructed from dsDNA isolated from cell-free DNA. In certain embodiments, the dsDNA can be end-repaired and A-tailed (ERAT) to produce 5′-phosphorylated, 3′-dA-tailed dsDNA fragments. After ERAT, dsDNA unique dual index adapters with 3′-dTMP overhangs can be ligated to 3′-dA-tailed dsDNA fragments. Indices allow for sample multiplex for the downstream assay. Post-ligation, a solid-phase reversible immobilization (SPRI) selection can be done to remove unwanted DNA fragments, excess adapters and molecules. PCR amplification can be performed, for example, with a high-fidelity, low-bias polymerase at about 10 or more cycles. Post-PCR, a SPRI selection can be done to remove unwanted DNA fragments, excess primers, excess adapters and excess molecules. After library construction, the library quality and quantity can be evaluated using the Agilent TapeStation and Qubit Fluorometer, respectively.

Libraries can be subjected to quality control checks prior to undergoing target enrichment, for example, through hybridization capture. Target enrichment by hybridization capture is a positive selection strategy to enrich low abundance regions of interest from NGS libraries, allowing for more accurate sequencing analysis of these target regions. Indexed libraries can be multiplexed and hybridized to a sequence specific, biotinylated probe set. The vast excess of probes drives their hybridization to complementary library fragments. The library fragment-biotinylated probe hybrid is pulled down by streptavidin beads, thereby capturing the target regions of interest. The streptavidin bead-bound library is sequentially washed with buffers to remove non-specifically associated library fragments. Following washes and recovery of captured libraries, samples are enriched for on target fragments and depleted for off-target fragments. Depletion of off-target fragments reduces overall library yield, requiring post-capture library amplification by PCR. The final amplified library is enriched for regions of interest. The hybrid captured library quality and quantity is evaluated, for example, using the Agilent TapeStation and Qubit Fluorometer, respectively. Additionally, the enrichment efficiency can be evaluated using an iSeq Sequencing run and calculation of percent of reads within target enrichment panel. Measuring percent on-target can serve as a first approximation of target enrichment efficiency because the reads aligning to the target enrichment (bait) region indicate efficient hybridization and subsequent capture.

Target enriched libraries can be subjected to quality control checks prior to undergoing NovaSeq sequencing. Captured libraries with non-overlapping indices from library construction can be pooled to multiplex for sequencing. Sequencing can be performed on a sequencer, such as the NovaSeq 6000 instrument using paired end 150×150 base sequencing with a 10% PhiX spike-in. Sequencing data generated can then be demultiplexed utilizing the assigned index, aligned to the human genome and trimmed to enrich for insert sample data only. This cleaned-up data can be processed to collapse duplicate reads and evaluate the sequencing data generated. Once the data is collapsed, the data is analyzed to identify differences from the reference alignment (e.g., mutations, chemical modifications such as methylation, etc.). A report can be generated with the specific biomarker analysis per sample that confirms the results of the phase 1 assay or identifies true false positives from the phase 1 assay. A second report can identify samples and specific biomarkers as potential neoantigens for therapeutic development (e.g., CDx development).

In certain embodiments, samples that are phase 1 (screening) positive, phase 2 (confirming) positive and are identified as having potential neoantigens can be further analyzed to detect neoantigens.

### III. Methods of Detecting Neoantigens

The disclosure relates to methods for detecting neoantigens that can be used as cancer vaccines, for example, personalized of universal cancer vaccines. After circulating tumor DNA in a sample from the subject is detected and the subject has been determined to be in need of a cancer vaccine, one or more neoantigens may be detected. The method can include obtaining nucleic acid sequence information from the subject, wherein the nucleic acid sequence information is used to obtain a peptide sequence of a neoantigen identified by comparing the nucleic acid sequence information from the subject to reference nucleic acid sequence information from normal cells, wherein the peptide sequence of the neoantigen comprises at least one sequence modification that distinguishes it from the corresponding peptide sequence identified from the reference nucleic acid sequence information. Reference nucleic acid information can be obtained from a sequence database or from a reference sample from the subject, wherein the reference sample is unlikely to or does not contain the neoantigen (e.g., a non-cancerous tissue).

### A. Neoantigens

Neoantigens are derived from somatic mutations in the tumor cell genome. In certain embodiments, a neoantigen comprises at least one sequence modification that distinguishes it from the corresponding peptide sequence identified from a reference nucleic acid sequence information. The sequence modification can be a gene fusion, a deletion, a frameshift mutation, a splice site mutation, a read-through mutation, or an amino acid substitution. Sequence modifications can also include one or more of nonframeshift indel, missense or nonsense substitution, or any genomic or expression alteration giving rise to a new open reading frame.

In certain embodiments, the sequence modification is a deletion in chromosome 17p. In certain embodiments, the deletion in chromosome 17p is a deletion in Eif5a and/or Alox15b/Alox8. In certain embodiments, the sequence modification disrupts a TP53 tumor suppressor gene.

In certain embodiments, the neoantigen is derived from a random somatic mutation specific to an individual, for example, the neoantigen is not common amongst a population of subjects (e.g., a neoantigen that can be used to produce a personalized cancer vaccine). In certain embodiments, the neoantigen is derived from a non-random mutation that is shared amongst a population of subjects (e.g., a neoantigen that can be used to produce a universal cancer vaccine).

### B. Methods of Obtaining Nucleic Acid Sequence Information

Nucleic acid sequence information can be obtained from a subject identified as being in need of a cancer vaccine. The nucleic acid sequence information is used to obtain a peptide sequence of a neoantigen identified by comparing the nucleic acid sequence information from the subject to reference nucleic acid sequence information from normal cells, wherein the peptide sequence of the neoantigen comprises at least one sequence modification that distinguishes it from the corresponding peptide sequence identified from the reference nucleic acid sequence information. Reference nucleic acid information can be obtained from a sequence database or from a reference sample from the subject, wherein the reference sample is unlikely to or does not contain the neoantigen (e.g., a non-cancerous tissue).

In certain embodiments, nucleic acid sequence information is obtained using a non-invasive assay that preferably uses small amounts of a target specimen from a sample. In certain embodiments, DNA and/or RNA from exosomes is used in the assay to obtain nucleic acid sequence information.

In some embodiments, nucleic acid sequence information relating to neoantigens can be detected in a sample from a subject. Examples of neoantigen-specific assays include, but are not limited to, nucleic acid amplification assays such as PCR assays, real-time PCR assays, quantitative real-time PCR (qPCR) assays, allele-specific PCR assays, and reverse-transcription PCR assays and protein detection assays such as ELISA assays and reporter assays. Nucleic acid amplification assays can include or be followed by nucleic acid sequencing, such as long-read sequencing.

### i. Mutation Detection Assays

A variety of methods are available for detecting the presence of a particular mutation or allele in an individual's DNA or RNA. Advancements in this field have provided accurate, easy, and inexpensive large-scale SNP genotyping. For example, several techniques have been described including dynamic allele-specific hybridization (DASH), microplate array diagonal gel electrophoresis (MADGE), pyrosequencing, oligonucleotide-specific ligation, the TaqMan system as well as various DNA “chip” technologies such as the Affymetrix SNP chips. These methods utilize amplification of a target genetic region, typically by PCR. Still other methods, based on the generation of small signal molecules by invasive cleavage followed by mass spectrometry or immobilized padlock probes and rolling circle amplification. Several of the methods known in the art for detecting specific mutations are summarized below.

PCR based detection means can include multiplex amplification of a plurality of markers simultaneously. For example, it is well known in the art to select PCR primers to generate PCR products that do not overlap in size and can be analyzed simultaneously. Alternatively, it is possible to amplify different markers with primers that are differentially labeled and thus can each be differentially detected. Of course, hybridization based detection means allow the differential detection of multiple PCR products in a sample. Other techniques are known in the art to allow multiplex analyses of a plurality of markers.

Several methods have been developed to facilitate analysis of single nucleotide polymorphisms in genomic DNA or cellular RNA. For example, a single base polymorphism can be detected by using a specialized exonuclease-resistant nucleotide, as disclosed, e.g., in U.S. Pat. No. 4,656,127. According to the method, a primer complementary to the allelic sequence immediately 3′ to the polymorphic site is permitted to hybridize to a target molecule obtained from a particular animal or human. If the polymorphic site on the target molecule contains a nucleotide that is complementary to the particular exonuclease resistant nucleotide derivative present, then that derivative will be incorporated onto the end of the hybridized primer. Such incorporation renders the primer resistant to exonuclease, and thereby permits its detection. Since the identity of the exonuclease-resistant derivative of the sample is known, a finding that the primer has become resistant to exonucleases reveals that the nucleotide(s) present in the polymorphic site of the target molecule is complementary to that of the nucleotide derivative used in the reaction. This method has the advantage that it does not require the determination of large amounts of extraneous sequence data.

A solution-based method can be used for determining the identity of a nucleotide of a polymorphic site. (PCT Publication. No. WO91/02087). As in the method of U.S. Pat. No. 4,656,127, a primer is employed that is complementary to allelic sequences immediately 3′ to a polymorphic site. The method determines the identity of the nucleotide of that site using labeled dideoxynucleotide derivatives, which, if complementary to the nucleotide of the polymorphic site will become incorporated onto the terminus of the primer.

An alternative method, known as Genetic Bit Analysis or GBA is described by Goelet et al. (PCT Publication No. WO92/15712; “Goelet”). The method of Goelet uses mixtures of labeled terminators and a primer that is complementary to the sequence 3′ to a polymorphic site. The labeled terminator that is incorporated is thus determined by, and complementary to, the nucleotide present in the polymorphic site of the target molecule being evaluated. In contrast to the method described in PCT Publication No. WO91/02087) the method of Goelet can be a heterogeneous phase assay, in which the primer or the target molecule is immobilized to a solid phase.

Several primer-guided nucleotide incorporation procedures for assaying polymorphic sites in DNA have been described (Komher, et al., (1989) NUCL. ACIDS. RES. 17:7779-7784; Sokolov (1990) NUCL. ACIDS RES. 18:3671; Syvanen, A.-C., et al. (1990) GENOMICS 8:684-692: Kuppuswamy et al. (1991) PROC. NATL. ACAD. SCI. (U.S.A.) 88:1143-1147; Prezant et al. (1992) HUM MUTAT. 1:159-164; Ugozzoli et al. (1992) GATA 9:107-112; Nyren et al. (1993) ANAL. BIOCHEM. 208:171-175). These methods differ from GBA in that they utilize incorporation of labeled deoxynucleotides to discriminate between bases at a polymorphic site. In such a format, since the signal is proportional to the number of deoxynucleotides incorporated, polymorphisms that occur in runs of the same nucleotide can result in signals that are proportional to the length of the run (Syvanen, A.-C., et al. (1993) AMER. J HUM. GENET. 52:46-59).

Allele-specific real-time reverse-transcriptase PCR can be performed by synthesizing cDNA from RNA (see New England Biosystems. First Strand cDNA Synthesis (NEB #M0253) Protocol. www.neb.com/protocols/2016/04/26/first-strand-cdna-synthesis-standard-protocol-neb-m0253) then combining cDNA with PCR reagents and primers specific for target sequences (see Lang et al. (2011) J. MOL. DIAG 13 (1), 23-28). The primers are designed to have single-base discrimination between tumor and non-tumor sequences. Real-time PCR (or digital PCR) can be run for, e.g., 30-50 cycles and the output monitored for signal (e.g., a fluorescence signal) from amplified target DNA or probe sequence. Cycle threshold values (Ct) can be recorded and exported for analysis. The delta-Ct between negative control, positive control, and sample can be calculated to determine presence or absence of target tumor sequences (see Mitchell et al. (2008) supra; Chubarov et al. (2020) supra; Fox et al. (1998) supra; Wong et al. (2005) supra). Slight modifications of this protocol can allow for end-point PCR detection of RNA or DNA of tumor sequences.

An example protocol of a real-time quantitative RNA assay is as follows:

Perform reverse transcriptase to synthesize cDNA from RNA (adapted from New England Biosystems (see New England Biosystems. First Strand cDNA Synthesis (NEB #M0253) Protocol. www.neb.com/protocols/2016/04/26/first-strand-cdna-synthesis-standard-protocol-neb-m0253)
1. Combine 10X Isothermal Amplification Buffer, 10 mM dNTPs, 60uM Random Primer Mix, nuclease-free water and up to 1 ug of Template Total RNA at a pre-specified reaction volume.
2. Mix 10X and centrifuge to collect volume at the bottom of the well or tube.
3. Denature sample RNA and primer for 5 minutes at 65° C. Put on ice immediately after incubation.
4. Add 10X M-MuLV buffer, 200U/uL M-MuLV reverse transcriptase, 40U/uL RNase inhibitor and nuclease water at a pre-specified reaction volume.
5. Mix 10X and centrifuge to collect volume at the bottom of the well or tube.
6. Incubate cDNA synthesis reaction at 25° C. for 5 minutes followed by 42° C. for 1 hour. Inactivate enzyme by incubating at 65° C. for 20 minutes.
7. Store at −20° C. Perform real-time quantitative PCR using hybridization of allele-specific probes (see Lang et al. (2011) J. MOL. DIAG: 13 (1), 23-28; Mitchell et al. (2008) PNAS:105 (30), 10513-10518; Chubarov et al. (2020). DIAGNOSTICS: 10, 872-886; Fox et al. (1998). BRITISH J. CANCER: 77(8), 1267-1274; Wong et al. (2005) BIOTECHNIQUES 39:1, 75-85)
8. This assay runs all cDNA samples in triplicate with 2 ng input in 5 uL for the reference and mutation assays.
9. Combine 900 nmol/L unspecific primer(s), 100 nmol/L target probe(s), 2X polymerase enzyme(s), 2X dNTPs, 2X passive reference dyes, 10 uL water and 2 ng sample DNA at a pre-specified reaction volume as the reference control assay.
10. Combine 450 nmol/L allele-specific primer(s), 100 nmol/L target probe(s), 2X polymerase enzyme(s), 2X dNTPs, 2X passive reference dyes, 10 uL water and 2 ng sample DNA at a pre-specified reaction volume as the mutation assay.
11. Mix each reaction 10X and centrifuge to collect volume at the bottom of the well or tube.
12. Run the real-time PCR on a calibrated Real-Time PCR system under the following conditions: (1) 95° C. for 10 minutes followed by (2) 50 cycles of 90° C. for 15 seconds and 60° C. for 1 minute with fluorescence detection using FAM/VIC fluorophores.
13. Cycle threshold (Ct) values are recorded by the system and exported into an analysis program (e.g., Excel).
14. Average the Ct values between sample replicates for the reference and mutation assays.
15. Calculate the DCt between the sample average allele-specific Ct minus the sample average unspecific (reference) Ct.
16. Positive mutation results are identified by the DCt cut off >3 cycles and will move forward to phase 2 testing.

### ii. Sequencing Methods

A number of initiatives obtain sequence information directly from millions of individual molecules of DNA or RNA in parallel. Real-time single molecule sequencing-by-synthesis technologies rely on the detection of fluorescent nucleotides as they are incorporated into a nascent strand of DNA that is complementary to the template being sequenced. In one method, oligonucleotides 30-50 bases in length are covalently anchored at the 5′ end to glass cover slips. These anchored strands perform two functions. First, they act as capture sites for the target template strands if the templates are configured with capture tails complementary to the surface-bound oligonucleotides. They also act as primers for the template directed primer extension that forms the basis of the sequence reading. The capture primers function as a fixed position site for sequence determination using multiple cycles of synthesis, detection, and chemical cleavage of the dye-linker to remove the dye. Each cycle consists of adding the polymerase/labeled nucleotide mixture, rinsing, imaging and cleavage of dye. In an alternative method, polymerase is modified with a fluorescent donor molecule and immobilized on a glass slide, while each nucleotide is color-coded with an acceptor fluorescent moiety attached to a gamma-phosphate. The system detects the interaction between a fluorescently-tagged polymerase and a fluorescently modified nucleotide as the nucleotide becomes incorporated into the de novo chain. Other sequencing-by-synthesis technologies also exist.

Any suitable sequencing-by-synthesis platform can be used to identify mutations. As described above, four major sequencing-by-synthesis platforms are the Genome Sequencers from Roche/454 Life Sciences, the 1G Analyzer from Illumina/Solexa, the SOLID system from Applied BioSystems, and the Heliscope system from Helicos Biosciences. Sequencing-by-synthesis platforn1s have also been described by Pacific BioSciences and VisiGen Biotechnologies. In some embodiments, a plurality of nucleic acid molecules being sequenced is bound to a support (e.g., solid support). To immobilize the nucleic acid on a support, a capture sequence/universal priming site can be added at the 3′ and/or 5′ end of the template. The nucleic acids can be bound to the support by hybridizing the capture sequence to a complementary sequence covalently attached to the support. The capture sequence (also referred to as a universal capture sequence) is a nucleic acid sequence complementary to a sequence attached to a support that may dually serve as a universal primer.

As an alternative to a capture sequence, a member of a coupling pair (such as, e.g., antibody/antigen, receptor/ligand, or the avidin-biotin pair as described in, e.g., U.S. Patent Publication No. 2006/0252077) can be linked to each fragment to be captured on a surface coated with a respective second member of that coupling pair. Subsequent to the capture, the sequence can be analyzed, for example, by single molecule detection/sequencing, e.g., as described in the Examples and in U.S. Pat. No. 7,283,337, including template-dependent sequencing-by-synthesis. In sequencing-by-synthesis, the surface-bound molecule is exposed to a plurality of labeled nucleotide triphosphates in the presence of polymerase. The sequence of the template is determined by the order of labeled nucleotides incorporated into the 3′ end of the growing chain. This can be done in real time or can be done in a step-and-repeat mode. For real-time analysis, different optical labels to each nucleotide can be incorporated and multiple lasers can be utilized for stimulation of incorporated nucleotides.

Sequencing can also include other massively parallel sequencing or next generation sequencing (NGS) techniques and platforms. Additional examples of massively parallel sequencing techniques and platforms are the Illumina HiSeq® or MiSeq®, Thermo Ion PGM™ or Ion Proton™, the PacBio RS II or Sequel, Qiagen's Gene Reader, and the Oxford Nanopore MinION®. Additional similar current massively parallel sequencing technologies can be used, as well as future generations of these technologies.

In some embodiments, nucleic acid sequence information relating to neoantigens can be detected in a sample from a subject (e.g., in plasma) using long-read nucleic acid sequencing. Use of long-read sequencing may be advantageous in certain instances because it avoids fragmentation that occurs during RNA sequencing methods that may preclude detection of fusions and other structural variants. For example, long-read RNA sequencing of nucleic acid from a biological sample can be performed to identify structural variants (fusions, deletions, insertions, etc.) that may form a neoantigen. In certain embodiments, exosomes are isolated from a biological sample, such as blood, and long-read sequencing of the mRNA from the exosome is performed to detect any structural variants (fusions, deletions, insertions, etc.) that may form a neoantigen. Long-read sequencing can be performed using, for example, an Oxford Nanopore instrument (e.g., PromethION™) or Pacific Biosciences Single-Molecule Real-Time (SMRT) sequencing technology. Methods of isolating exosomes are known in the art. See, for example, Aziz et al. (2022) J NUCLEIC ACIDS Article ID 8648373.

Samples that have specific neoantigen(s) detected can be candidates for mRNA cancer vaccines.

### IV. Neoantigen Cancer Vaccine Compositions and Methods of Making Same

In another aspect, the disclosure relates to a method of producing a peptide or polynucleotide neoantigen cancer vaccine, comprising obtaining nucleic acid sequence information from a subject determined to have circulating tumor DNA, wherein the nucleic acid sequence information is used to obtain a peptide sequence of a neoantigen identified by comparing the nucleic acid sequence information from the subject to reference nucleic acid sequence information from normal cells, wherein the peptide sequence of the neoantigen comprises at least one sequence modification that distinguishes it from the corresponding peptide sequence identified from the reference nucleic acid sequence information; and producing the peptide neoantigen cancer vaccine comprising the peptide sequence of the neoantigen or the polynucleotide neoantigen cancer vaccine encoding the peptide sequence of the neoantigen.

Proteins or peptides can be made by any technique known to those of skill in the art, including the expression of proteins, polypeptides or peptides through standard molecular biological techniques, the isolation of proteins or peptides from natural sources, or the chemical synthesis of proteins or peptides. The nucleotide and protein, polypeptide and peptide sequences corresponding to various genes have been previously disclosed, and can be found at computerized databases known to those of ordinary skill in the art. One such database is the National Center for Biotechnology Information's Genbank and GenPept databases located at the National Institutes of Health Website. The coding regions for known genes can be amplified and/or expressed using the techniques disclosed herein or as would be known to those of ordinary skill in the art. Alternatively, various commercial preparations of proteins, polypeptides and peptides are known to those of skill in the art.

In a further aspect a neoantigen includes a nucleic acid (e.g., polynucleotide) that encodes a neoantigenic peptide or portion thereof. The polynucleotide can be, e.g., DNA, cDNA PNA, CNA, RNA (e.g., mRNA), either single-and/or double-stranded, or native or stabilized forms of polynucleotides, such as, e.g., polynucleotides with a phosphorothiate backbone, or combinations thereof and it may or may not contain introns. A still further aspect provides an expression vector capable of expressing a polypeptide or portion thereof. Expression vectors for different cell types are well known in the art and can be selected without undue experimentation. Generally, DNA is inserted into an expression vector, such as a plasmid, in proper orientation and correct reading frame for expression. If necessary, DNA can be linked to the appropriate transcriptional and translational regulatory control nucleotide sequences recognized by the desired host, although such controls are generally available in the expression vector. The vector is then introduced into the host through standard techniques. Guidance can be found, e.g., in Sambrook et al. (2001), supra.

Methods for generating a vaccine using a neoantigen as described herein can be found in the art, for example, in Jackson et al., (2020) NPJ VACCINES 5, 11. In certain embodiments, a vaccine is generated by the following steps. First, plasmid DNA containing a DNA-dependent RNA polymerase and target sequence for an mRNA construct is generated. Next, plasmid DNA is linearized, transcribed to mRNA and degraded by a DNase step. After transcription, the 5′ cap and 3′ poly-A tail are enzymatically added. The mRNA construct is then purified using HPLC. Self-amplifying mRNA (SAM) is packaged in lipid nanoparticles. Cellular uptake of the mRNA is ensured by utilizing a membrane-derived endocytic pathway. Using endosomal escape, mRNA is released into the cytosol of an immune cell. SAMs are translated by ribosome to produce replicase components able to direct intracellular self-mRNA amplification. Self-amplified mRNA constructs are translated by ribosomes to produce the protein of interest (e.g., neoantigen), which undergoes subsequent post-translational modification. Expressed protein of interest (e.g., neoantigen) is generated as a secreted, trans-membrane intracellular protein. The innate and adaptive immune responses detect the protein of interest, thereby vaccinating the subject against cancer.

A vaccine composition can further comprise an adjuvant and/or a carrier. Examples of useful adjuvants and carriers are given herein below. A composition can be associated with a carrier such as e.g. a protein or an antigen-presenting cell such as e.g. a dendritic cell (DC) capable of presenting the peptide to a T-cell.

Adjuvants are any substance whose admixture into a vaccine composition increases or otherwise modifies the immune response to a neoantigen. Carriers can be scaffold structures, for example a polypeptide or a polysaccharide, to which a neoantigen, is capable of being associated. Optionally, adjuvants are conjugated covalently or noncovalently.

The ability of an adjuvant to increase an immune response to an antigen is typically manifested by a significant or substantial increase in an immune-mediated reaction, or reduction in disease symptoms. For example, an increase in humoral immunity is typically manifested by a significant increase in the titer of antibodies raised to the antigen, and an increase in T-cell activity is typically manifested in increased cell proliferation, or cellular cytotoxicity, or cytokine secretion. An adjuvant may also alter an immune response, for example, by changing a primarily humoral or Th response into a primarily cellular, or Th response.

Suitable adjuvants include, but are not limited to 1018 ISS, alum, aluminum salts, Amplivax, AS15, BCG, CP-870,893, CpG-7909, CyaA, dSLIM, GM-CSF, IC30, IC31, Imiquimod, ImuFact IMP321, IS Patch, ISS, ISCOMATRIX, JuvImmune, Lipo Vac, MF59, monophosphoryl lipid A, Montanide IMS 1312, Montanide ISA 206, Montanide ISA 50V, Montanide ISA-51, OK-432, OM-174, OM-197-MP-EC, ONTAK, PepTel vector system, PLG microparticles, resiquimod, SRL 172, Virosomes and other Virus-like particles, YF-17D, VEGF trap, R848, beta-glucan, Pam3Cys, Aquila's QS21 stimulon (Aquila Biotech, Worcester, Mass., USA) which is derived from saponin, mycobacterial extracts and synthetic bacterial cell wall mimics, and other proprietary adjuvants such as Ribi's Detox. Quil or Superfos. Adjuvants such as incomplete Freund's or GM-CSF may be useful. Several immunological adjuvants (e.g., MF59) specific for dendritic cells and their preparation have been described previously (Dupuis et al. (1998) CELL IMMUNOL. 86(1): 18-27: Allison (1998) DEV BIOL STAND. 92:3-11). Also cytokines can be used. Several cytokines have been directly linked to influencing dendritic cell migration to lymphoid tissues (e.g., TNF-alpha), accelerating the maturation of dendritic cells into efficient antigen-presenting cells for T-lymphocytes (e.g., GM-CSF, IL-1 and IL-4) (U.S. Pat. No. 5,849,589, incorporated herein by reference in its entirety) and acting as immunoadjuvants (e.g., IL-12) (Gabrilovich et al. (1996) J IMMUNOTHER EMPHASIS TUMOR IMMUNOL. (6):414-418).

CpG immunostimulatory oligonucleotides have also been reported to enhance the effects of adjuvants in a vaccine setting. Other TLR binding molecules such as RNA binding TLR 7, TLR 8 and/or TLR 9 may also be used.

Other examples of useful adjuvants include, but are not limited to, chemically modified CpGs (e.g. CpR, Idera), Poly (I:C) (e.g., polyi:CI2U), non-CpG bacterial DNA or RNA as well as immunoactive small molecules and antibodies such as cyclophosphamide, sunitinib, bevacizumab, celebrex, NCX-4016, sildenafil, tadalafil, vardenafil, sorafinib, XL-999, CP-547632, pazopanib, ZD217 1, AZD217L ipilimumab, tremelimumab, and SC58175, which may act therapeutically and/or as an adjuvant. The amounts and concentrations of adjuvants and additives can readily be determined by the skilled artisan without undue experimentation. Additional adjuvants include colony-stimulating factors, such as Granulocyte Macrophage Colony Stimulating Factor (GM-CSF, sargramostim).

A vaccine composition can comprise more than one different adjuvant. Furthermore, a therapeutic composition can comprise any adjuvant substance including any of the above or combinations thereof. It is also contemplated that a vaccine and an adjuvant can be administered together or separately in any appropriate sequence.

A carrier (or excipient) can be present independently of an adjuvant. The function of a carrier can for example be to increase the molecular weight of in particular mutant to increase activity or immunogenicity, to confer stability, to increase the biological activity, or to increase serum half-life. Furthermore, a carrier can aid presenting peptides to T-cells. A carrier can be any suitable carrier known to the person skilled in the art, for example a protein or an antigen presenting cell. A carrier protein could include, but is not limited to, keyhole limpet hemocyanin, serum proteins such as transferrin, bovine serum albumin, human serum albumin, thyroglobulin or ovalbumin, immunoglobulins, or hormones, such as insulin or palmitic acid. For immunization of humans, the carrier is generally a physiologically acceptable carrier acceptable to humans and safe. However, tetanus toxoid and/or diphtheria toxoid are suitable carriers. Alternatively, the carrier can be dextrans, for example, a cross-linked agarose such as Sepharose™.

In certain embodiments, a candidate neoantigen vaccine is tested for the ability to bind to the subject's HLA prior to administration of the vaccine, for example, by using a prediction algorithm or testing for binding experimentally (see, e.g., methods described in U.S. Pat. No. 9,115,402 and WO2017/106638, each of which is incorporated by reference herein in their entireties).

### V. Methods of Administering Cancer Vaccines

The disclosure further relates to methods for vaccinating a subject against cancer using the neoantigens detected according to the methods described herein. Such neoantigens can be administered to the subject such that the neoantigen is presented to the subject's immune system, thereby vaccinating the subject against cancer. In certain embodiments, the neoantigen cancer vaccine comprises a nucleotide sequence, such as a DNA or and RNA (e.g., an mRNA), a protein or peptide sequence, a cell, a plasmid, or a vector.

The optimum amount of each neoantigen to be included in a vaccine composition and the optimum dosing regimen can be determined. For example, a neoantigen or its variant can be prepared for intravenous injection, sub-cutaneous injection, intradermal injection, intraperitoneal injection, intramuscular injection. Methods of injection include intravenous, sub-cutaneous, intradermal, intraperitoneal, and intramuscular. Methods of DNA or RNA injection include intravenous, sub-cutaneous, intradermal, intraperitoneal, and intramuscular. Other methods of administration of the vaccine composition are known to those skilled in the art. Compositions comprising a neoantigen can be administered to an individual already suffering from cancer. In therapeutic applications, compositions are administered to a subject in an amount sufficient to elicit an effective immune response to the tumor antigen and to cure or at least partially arrest symptoms and/or complications. An amount adequate to accomplish this is defined as “therapeutically effective dose.” Amounts effective for this use will depend on, e.g., the composition, the manner of administration, the stage and severity of the disease being treated, the weight and general state of health of the subject, and the judgment of the prescribing physician.

For therapeutic use, administration can begin at the detection or surgical removal of tumors. This can be followed by boosting doses until at least symptoms are substantially abated and for a period thereafter. The pharmaceutical compositions (e.g., vaccine compositions) for therapeutic treatment are intended for parenteral, topical, nasal, oral, or local administration. A pharmaceutical composition can be administered parenterally, e.g., intravenously, subcutaneously, intradermally, or intramuscularly. The compositions can be administered at the site of surgical excision to induce a local immune response to the tumor. Disclosed herein are compositions for parenteral administration which comprise a solution of the neoantigen and vaccine compositions are dissolved or suspended in an acceptable carrier, e.g., an aqueous carrier. A variety of aqueous carriers can be used, e.g., water, buffered water, 0.9% saline, 0.3% glycine, hyaluronic acid and the like. These compositions can be sterilized by conventional, well known sterilization techniques, or can be sterile filtered. The resulting aqueous solutions can be packaged for use as is, or lyophilized, the lyophilized preparation being combined with a sterile solution prior to administration. The compositions may contain pharmaceutically acceptable auxiliary substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. Neoantigens can also be administered via liposomes, which target them to a particular cells tissue, such as lymphoid tissue. Liposomes are also useful in increasing half-life. Liposomes include emulsions, foams, micelles, insoluble monolayers, liquid crystals, phospholipid dispersions, lamellar layers and the like. In these preparations the neoantigen to be delivered is incorporated as part of a liposome, alone or in conjunction with a molecule which binds to, e.g., a receptor prevalent among lymphoid cells, such as monoclonal antibodies which bind to the CD45 antigen, or with other therapeutic or immunogenic compositions. Thus, liposomes filled with a desired neoantigen can be directed to the site of lymphoid cells, where the liposomes then deliver the selected therapeutic/immunogenic compositions. Liposomes can be formed from standard vesicle-forming lipids, which generally include neutral and negatively charged phospholipids and a sterol, such as cholesterol. The selection of lipids is generally guided by consideration of, e.g., liposome size, acid lability and stability of the liposomes in the blood stream. A variety of methods are available for preparing liposomes, as described in, e.g., Szoka et al. (1980) ANN. REV. BIOPHYS. BIOENG. 9; 467 (1980), U.S. Pat. Nos. 4,235,871, 4,501,728, 4,501,728, 4,837,028, and 5,019,369.

For targeting to the immune cells, a ligand to be incorporated into the liposome can include, e.g., antibodies or fragments thereof specific for cell surface determinants of the desired immune system cells. A liposome suspension can be administered intravenously, locally, topically, etc. in a dose which varies according to, inter alia, the manner of administration, the peptide being delivered, and the stage of the disease being treated.

For therapeutic or immunization purposes, nucleic acids encoding a peptide and optionally one or more of the peptides described herein can also be administered to the subject. A number of methods can be used to deliver the nucleic acids to the subject. For instance, the nucleic acid can be delivered directly, as “naked DNA”. This approach is described, for instance, in Wolff et al., Science 247: 1465-1468 (1990) as well as U.S. Pat. Nos. 5,580,859 and 5,589,466. The nucleic acids can also be administered using ballistic delivery as described, for instance, in U.S. Pat. No. 5,204,253. Particles comprised solely of DNA can be administered. Alternatively, DNA can be adhered to particles, such as gold particles. Approaches for delivering nucleic acid sequences can include viral vectors, mRNA vectors, and DNA vectors with or without electroporation.

The nucleic acids can also be delivered complexed to cationic compounds, such as cationic lipids. Lipid-mediated gene delivery methods are described, for instance, in WO 96/18372, WO 93/24640; Mannino & Gould-Fogerite (1988) BIOTECHNIQUES 6(7): 682-691;U.S. Pat. Nos. 5,279,833, and 5,279,833. Also disclosed is a method of manufacturing a tumor vaccine, comprising performing the steps of a method disclosed herein; and producing a tumor vaccine comprising a plurality of neoantigens or a subset of the plurality of neoantigens.

Neoantigens disclosed herein can be manufactured using methods known in the art. For example, a method of producing a neoantigen or a vector (e.g., a vector including at least one sequence encoding one or more neoantigens) disclosed herein can include culturing a host cell under conditions suitable for expressing the neoantigen or vector wherein the host cell comprises at least one polynucleotide encoding the neoantigen or vector and purifying the neoantigen or vector. Standard purification methods include chromatographic techniques, electrophoretic, immunological, precipitation, dialysis, filtration, concentration, and chromatofocusing techniques.

Throughout the description, where apparatus, devices, and systems are described as having, including, or comprising specific components, or where processes and methods are described as having, including, or comprising specific steps, it is contemplated that, additionally, there are apparatus, devices, and systems of the present invention that consist essentially of, or consist of, the recited components, and that there are processes and methods according to the present invention that consist essentially of, or consist of, the recited processing steps.

Practice of the invention will be more fully understood from the foregoing examples, which are presented herein for illustrative purposes only, and should not be construed as limiting the invention in any way.

### EXAMPLES

### Example 1—Detection of Neoantigens for Cancer Vaccines

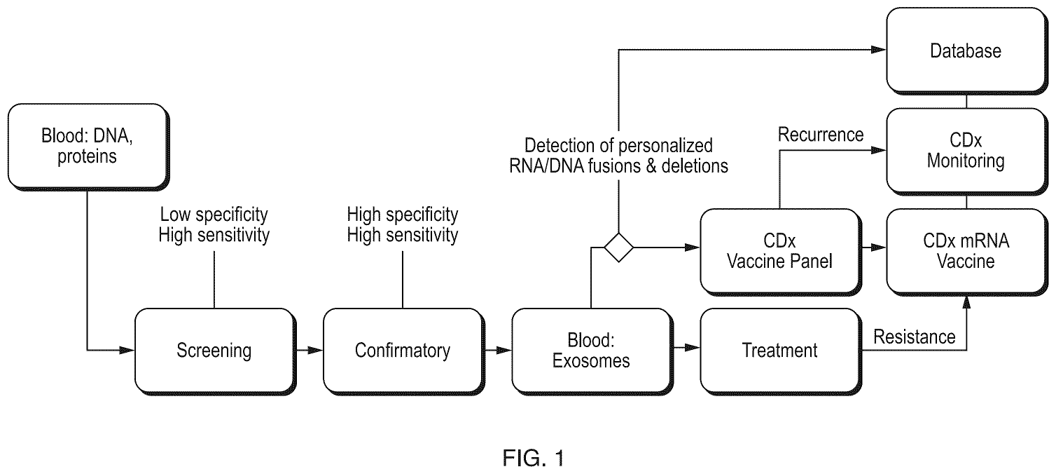

This example describes a process for detecting neoantigens for personalized or universal cancer vaccines (see, e.g., FIG. 1).

### Prepare Target Specimen

The target specimen type (e.g., DNA, RNA, protein, exosomes, metabolites, etc.) is isolated from a subject sample (e.g., tissue, blood, plasma, serum, saliva, feces, etc.). (See Yamagata et al. (2021) SCI REP 11, 17075 (2021); Zeringer et al. (2013) WORLD J METHODOL. 3(1):11-8. doi: 10.5662/wjm.v3.il.11. PMID: 25237619; PMCID: PMC4145569; Macherey-Nagel. RNA from blood. NucleoSpin® RNA Blood Kit. User Manual. www.mn-net.com/media/pdf/0d/f8/93/Instruction-NucleoSpin-RNA-Blood.pdf; Qiagen. DNA from blood. QIAamp® DNA Blood Kit. User Manual. file:///Users/cgiley/Downloads/EN-QIAamp-96-DNA-Blood-Handbook.pdf; Qiagen. Exosomal RNA from blood. exoRNeasy Midi/Maxi Handbook. https://www.qiagen.com/us/products/discovery-and-translational-research/exosomes-ctcs/exosomes/exorneasy-midi-and-maxi-kits/. See also example protocols in FIGS. 3 and 4.) All target specimens may be assayed for quality and quantity measurements. Phase 1 (Screen) testing

Phase 1 testing is a quick, non-invasive assay, using small amounts of the target specimen (e.g., DNA, RNA, protein, etc.). The result of this assay can be both qualitative and quantitative. Phase 1 testing is typically lower specificity (e.g., 95% specificity, 5% false positives) but higher sensitivity (e.g., 80% sensitivity, 20% false negatives) in order to screen a large proportion of the testing population rapidly and inexpensively.

Examples of the Phase 1 assay include but are not limited to Bradford protein assays, ELISA assays, PCR assays, Real-time PCR assays, Quantitative real-time PCR (qPCR) assays, Allele-specific PCR assays, Reverse-transcription PCR assays and reporter assays.

An example protocol of an Allele-specific Real-Time PCR (see Lang et al. (2011) J. MOL. DIAG: 13 (1), 23-28; Mitchell et al. (2008) PNAS: 105 (30), 10513-10518; Chubarov et al. (2020). DIAGNOSTICS: 10, 872-886; Fox et al. (1998). BRITISH J. CANCER: 77(8),1267-1274; Wong et al. (2005) BIOTECHNIQUES 39:1, 75-85) assay is as follows:
1. This assay runs all DNA samples in triplicate with 2 ng input in 5 uL for the reference and mutation assays.
2. Combine 900 nmol/L unspecific primer(s), 100 nmol/L target probe(s), 2X polymerase enzyme(s), 2X dNTPs, 2X passive reference dyes, 10 uL water and 2 ng sample DNA at a pre-specified reaction volume as the reference control assay.
3. Combine 450 nmol/L allele-specific primer(s), 100 nmol/L target probe(s), 2X polymerase enzyme(s), 2X dNTPs, 2X passive reference dyes, 10 uL water and 2 ng sample DNA at a pre-specified reaction volume as the mutation assay.
4. Mix each reaction 10X and centrifuge to collect volume at the bottom of the well or tube.
5. Run the real-time PCR on a calibrated Real-Time PCR system under the following conditions: (1) 95° C. for 10 minutes followed by (2) 50 cycles of 90° C. for 15 seconds and 60° C. for 1 minute with fluorescence detection using FAM/VIC fluorophores.
6. Cycle threshold (Ct) values are recorded by the system and exported into an analysis program (e.g. Excel).
7. Average the Ct values between sample replicates for the reference and mutation assays.
8. Calculate the DCt between the sample average allele-specific Ct minus the sample average unspecific (reference) Ct.
9. Positive mutation results are identified by the DCt cut off >3 cycles and will move forward to phase 2 testing.

Allele-specific real-time PCR can be performed by combining library DNA with PCR reagents and primers specific for target sequences (see Lang et al. (2011), supra). The primers are designed to have single-base discrimination between tumor and non-tumor sequences. Perform real-time PCR (or digital PCR) for 30-50 cycles and monitor the output for signal via fluorescence from amplified target DNA or probe sequence. Cycle threshold values (Ct) are recorded and exported for analysis. The delta-Ct between negative control, positive control, and sample are calculated to determine presence or absence of target tumor sequences (see Mitchell et al. (2008) supra; Chubarov et al. (2020) supra; Fox et al. (1998) supra; Wong et al. (2005) supra). Slight modifications of this protocol will allow for end-point PCR detection of RNA or DNA of tumor sequences.

ELISA assay detection of target molecules can be performed by coating an immunoassay well with monoclonal antibody designed to specifically detect target molecules (see Corey et al. (1997) INT. J. CANCER 71,1019-1028), followed by blocking against non-specific binding. Next, target sample is introduced to the well, incubated and washed away. Any bound target can then be bound by a polyclonal antibody specific for the target. Additional secondary antibodies with color or fluorescent tags can be used to detect the presence of target molecules.

Samples that are Phase 1 (Screening) positive will move to Phase 2 (Confirmatory). Phase 1 detection will be designed to remove 90-95% of non-cancer patient samples from moving forward for further testing.

### Phase 2 (Confirm) Testing

Phase 2 testing is a more complex assay, potentially using larger amounts of the target specimen (e.g., DNA, RNA, protein, etc.). The result of this assay may be both qualitative and quantitative. Phase 2 testing is typically higher specificity (e.g., 90% specificity, 10% false positives) but lower sensitivity (e.g., 90% sensitivity, 10% false negatives) in order to limit false positives.

Examples of the Phase 2 assay include but are not limited to Next Generation Sequencing assays utilizing target enrichment technologies, targeted amplicon sequencing technologies, and/or whole genome sequencing.

The target specimen for library construction is dsDNA isolated from cell-free DNA. The dsDNA is then end-repaired and A-tailed (ERAT) to produce 5′-phosphorylated, 3′-dA-tailed dsDNA fragments. After ERAT, dsDNA unique dual index adapters with 3′-dTMP overhangs are then ligated to 3′-dA-tailed dsDNA fragments. Indices allow for sample multiplex for the downstream assay. Post-ligation, a solid-phase reversible immobilization (SPRI) selection is done to remove unwanted DNA fragments, excess adapters and molecules. PCR amplification is performed with a high-fidelity, low-bias polymerase at 10 cycles. Post-PCR, a SPRI selection is done to remove unwanted DNA fragments, excess primers, excess adapters and excess molecules. After library construction, the library quality and quantity are evaluated using the Agilent TapeStation and Qubit Fluorometer, respectively.

Libraries that pass all quality control checks move forward to target enrichment through hybridization capture. Target enrichment by hybridization capture is defined as a positive selection strategy to enrich low abundance regions of interest from NGS libraries, allowing for more accurate sequencing analysis of these target regions. Indexed libraries are multiplexed and hybridized to a custom, sequence specific, biotinylated probeset. The vast excess of probes drives their hybridization to complementary library fragments. The library fragment-biotinylated probe hybrid is pulled down by streptavidin beads, thereby capturing the target regions of interest. The streptavidin bead-bound library is sequentially washed with buffers to remove non-specifically associated library fragments. Following washes and recovery of captured libraries, samples are enriched for on target fragments and depleted for off-target fragments. Depletion of off-target fragments reduces overall library yield, requiring post-capture library amplification by PCR. The final amplified library is enriched for regions of interest. The hybrid captured library quality and quantity is evaluated using the Agilent TapeStation and Qubit Fluorometer, respectively. Additionally, the enrichment efficiency is evaluated using an iSeq Sequencing run and calculation of percent of reads within target enrichment panel. Measuring percent on-target is a good first approximation of target enrichment efficiency because the reads aligning to the target enrichment (bait) region indicate efficient hybridization and subsequent capture.

Target enriched libraries that pass all quality control checks move forward to NovaSeq sequencing. Captured libraries with non-overlapping indices from library construction are pooled to multiplex for sequencing. Sequencing is completed on the NovaSeq 6000instrument using paired end 150x150 base sequencing with a 10% PhiX spike-in. Sequencing data generated is then demultiplexed utilizing the assigned index, aligned to the human genome and trimmed to enrich for insert sample data only. This cleaned-up data is then processed through a quality pipeline to collapse duplicate reads and evaluate the sequencing data generated. Once the data is collapsed, the data is processed through a proprietary biomarker analysis pipeline to identify differences from the reference alignment (e.g., mutations, chemical modifications, etc.). A report is then generated with the specific biomarker analysis per sample that confirms the results of the phase 1 assay or identifies true false positives from the phase 1 assay. A separate report will identify samples and specific biomarkers as potential neoantigens for therapeutic development (e.g., CDx development).

Samples that are Phase 1 (Screening) positive, Phase 2 (Confirming) positive and are identified as having potential neoantigens will move to Neoantigen-specific testing.

### Neoantigen Specific Detection Testing

Neoantigen specific testing is a non-invasive assay using small amounts of the target specimen (e.g., DNA, RNA, protein, exosomes etc.). The result of this assay can be both qualitative and quantitative.

Examples of the Neoantigen specific assay include but are not limited to PCR assays, Real-time PCR assays, Quantitative real-time PCR (qPCR) assays, Allele-specific PCR assays, Reverse-transcription PCR assays, ELISA assays and reporter assays.

An example protocol of a real-time quantitative RNA assay is as follows: Perform reverse transcriptase to synthesize cDNA from RNA (adapted from New England Biosystems (see New England Biosystems. First Strand cDNA Synthesis (NEB #M0253) Protocol. www.neb.com/protocols/2016/04/26/first-strand-cdna-synthesis-standard-protocol-neb-m0253)
1. Combine 10X Isothermal Amplification Buffer, 10 mM dNTPs, 60 uM Random Primer Mix, nuclease-free water and up to 1 ug of Template Total RNA at a pre-specified reaction volume.
2. Mix 10X and centrifuge to collect volume at the bottom of the well or tube.
3. Denature sample RNA and primer for 5 minutes at 65° C. Put on ice immediately after incubation.
4. Add 10X M-MuLV buffer, 200U/uL M-MuLV reverse transcriptase, 40U/uL RNase inhibitor and nuclease water at a pre-specified reaction volume.
5. Mix 10X and centrifuge to collect volume at the bottom of the well or tube.
6. Incubate cDNA synthesis reaction at 25° C. for 5 minutes followed by 42° C. for 1 hour. Inactivate enzyme by incubating at 65° C. for 20 minutes.
7. Store at −20° C.

Perform real-time quantitative PCR using hybridization of allele-specific probes (see Lang et al. (2011) J. MOL. DIAG. 13 (1): 23-28; Mitchell et al. (2008) PNAS 105 (30): 10513-10518; Chubarov et al. (2020) DIAGNOSTICS: 10, 872-886; Fox et al. (1998) BRITISH J. CANCER: 77(8),1267-1274; Wong et al. (2005) BIOTECHNIQUES 39:1, 75-85)
8. This assay runs all cDNA samples in triplicate with 2 ng input in 5 uL for the reference and mutation assays.
9. Combine 900 nmol/L unspecific primer(s), 100 nmol/L target probe(s), 2X polymerase enzyme(s), 2X dNTPs, 2X passive reference dyes, 10 uL water and 2 ng sample DNA at a pre-specified reaction volume as the reference control assay.
10. Combine 450 nmol/L allele-specific primer(s), 100 nmol/L target probe(s), 2X polymerase enzyme(s), 2X dNTPs, 2X passive reference dyes, 10 uL water and 2 ng sample DNA at a pre-specified reaction volume as the mutation assay.
11. Mix each reaction 10X and centrifuge to collect volume at the bottom of the well or tube.
12. Run the real-time PCR on a calibrated Real-Time PCR system under the following conditions: (1) 95° C. for 10 minutes followed by (2) 50 cycles of 90° C. for 15 seconds and 60° C. for 1 minute with fluorescence detection using FAM/VIC fluorophores.
13. Cycle threshold (Ct) values are recorded by the system and exported into an analysis program (e.g., Excel).
14. Average the Ct values between sample replicates for the reference and mutation assays.
15. Calculate the DCt between the sample average allele-specific Ct minus the sample average unspecific (reference) Ct.
16. Positive mutation results are identified by the DCt cut off >3 cycles and will move forward to phase 2 testing.

Allele-specific real-time reverse-transcriptase PCR can be performed by synthesizing cDNA from RNA (see New England Biosystems. First Strand cDNA Synthesis (NEB #M0253) Protocol. www.neb.com/protocols/2016/04/26/first-strand-cdna-synthesis-standard-protocol-neb-m0253) then combining cDNA with PCR reagents and primers specific for target sequences (see Lang et al. (2011) J. MOL. DIAG 13 (1), 23-28). The primers are designed to have single-base discrimination between tumor and non-tumor sequences. Real-time PCR (or digital PCR) is performed for 30-50 cycles and the output is monitored for signal via fluorescence from amplified target DNA or probe sequence. Cycle threshold values (Ct) are recorded and exported for analysis. The delta-Ct between negative control, positive control, and sample are calculated to determine presence or absence of target tumor sequences (see Mitchell et al. (2008) supra; Chubarov et al. (2020) supra; Fox et al. (1998) supra; Wong et al. (2005) supra). Slight modifications of this protocol will allow for end-point PCR detection of RNA or DNA of tumor sequences.

Samples that are Phase 1 (Screening) positive, Phase 2 (Confirming) positive and have specific neoantigen(s) detected will then be candidate for mRNA cancer vaccines.

Creation of Personalized (or Universal) mRNA Vaccine Derived From Neoantigen Detection Assay

Using the neoantigen detection assay described above, identify personalized or universal expressed neoantigens (e.g., expressed RNA fusions, expressed deletions, etc.) and develop mRNA vaccines from personalized or universal neoantigens.

mRNA vaccine development adapted from Jackson et al., (2020) NPJ VACCINES 5, 11.
1.Generate plasmid DNA containing a DNA-dependent RNA polymerase and target sequence for mRNA construct.
2.Plasmid DNA is linearized, transcribed to mRNA and degraded by a DNase step.
3.After transcription, the 5′ cap and 3′ poly-A tail are enzymatically added.
4.The mRNA construct is then purified using HPLC.
5.Form self-amplifying mRNA (SAM) in lipid nanoparticles.
6.Ensure cellular uptake of the mRNA utilizing a membrane-derived endocytic pathway.
7.Using endosomal escape, release mRNA into cytosol of immune cell.
8. SAMs are immediately translated by ribosome to produce replicase components able to direct intracellular self-mRNA amplification.
9. Self-amplified mRNA constructs are translated by ribosomes to produce protein of interest, which undergoes subsequent post-translational modification.
10. Expressed protein of interest are generated as secreted, trans-membrane intracellular protein
11. The innate and adaptive immune responses detect the protein of interest

It is believed that a subject who is administered a personalized or universal cancer vaccine created by the foregoing method will experience a stimulated immune response against the tumor cells expressing the neoantigen, thereby reducing or eliminating the tumor.

### INCORPORATION BY REFERENCE

The entire disclosure of each of the patent and scientific documents referred to herein is incorporated by reference for all purposes.

### Equivalents

The invention may be embodied in other specific forms without departing from the spirit or essential characteristics thereof. The foregoing embodiments are therefore to be considered in all respects illustrative rather than limiting on the invention described herein. Scope of the invention is thus indicated by the appended claims rather than by the foregoing description, and all changes that come within the meaning and range of equivalency of the claims are intended to be embraced therein.

1-45. (canceled)

46. A method for vaccinating a subject against cancer, comprising:
detecting circulating tumor DNA in a sample from the subject to determine the subject is in need of a cancer vaccine;
obtaining nucleic acid sequence information from the subject, wherein the nucleic acid sequence information is used to obtain a peptide sequence of a neoantigen identified by comparing the nucleic acid sequence information from the subject to reference nucleic acid sequence information from normal cells, wherein the peptide sequence of the neoantigen comprises at least one sequence modification that distinguishes it from the corresponding peptide sequence identified from the reference nucleic acid sequence information; and
administering the neoantigen to the subject such that the neoantigen is presented to the subject's immune system, thereby vaccinating the subject against cancer.

47. The method of claim 46, wherein the sequence modification is gene fusion, a deletion, a frameshift mutation, a splice site mutation, a read-through mutation, or an amino acid substitution.

48. The method of claim 46, wherein the circulating tumor DNA originates from a precancerous cell or a cancerous cell.

49. The method of claim 46, wherein the circulating tumor DNA is detected using a methylome-based assay.

50. The method of claim 46, wherein the methylome-based assay analyzes methylation of a plurality of CpG sites within a chromosomal region selected from the CpG sites listed in Table 1 of PCT/US2018/032612 and Tables 2 and 3 of PCT/US2021/064210.

51. The method of claim 46, wherein the neoantigen is derived from a random somatic mutation specific to the subject or a non-random mutation that is shared amongst a population of subjects.

52. The method of claim 46—, wherein the sequence modification is a deletion in chromosome 17p in Eif5a and/or Alox15b/Alox8.

53. The method of claim 46, wherein the nucleic acid sequence information is obtained by long-read sequencing and wherein the nucleic acid sequence information is from mRNA or obtained by isolating nucleic acid derived from exosomes.

54. A method of producing a peptide or polynucleotide neoantigen cancer vaccine, comprising:
obtaining nucleic acid sequence information from a subject determined to have circulating tumor DNA, wherein the nucleic acid sequence information is used to obtain a peptide sequence of a neoantigen identified by comparing the nucleic acid sequence information from the subject to reference nucleic acid sequence information from normal cells, wherein the peptide sequence of the neoantigen comprises at least one sequence modification that distinguishes it from the corresponding peptide sequence identified from the reference nucleic acid sequence information; and
producing the peptide neoantigen cancer vaccine comprising the peptide sequence of the neoantigen or the polynucleotide neoantigen cancer vaccine encoding the peptide sequence of the neoantigen.

55. The method of claim 54, wherein the sequence modification is gene fusion, a deletion, a frameshift mutation, a splice site mutation, a read-through mutation, or an amino acid substitution.

56. The method of claim 54, wherein the circulating tumor DNA originates from a precancerous cell or a cancerous cell.

57. The method of claim 54, wherein a methylome-based assay was used to determine that the subject had the circulating tumor DNA.

58. The method of claim 54, wherein the methylome-based assay analyzes methylation of a plurality of CpG sites within a chromosomal region selected from the CpG sites listed in Table 1 of PCT/US2018/032612 and Tables 2 and 3 of PCT/US2021/064210.

59. The method of claim 54, wherein the neoantigen is derived from a random somatic mutation specific to the subject or a non-random mutation that is shared amongst a population of subjects.

60. The method of claim 54, wherein the sequence modification is a deletion in chromosome 17p in Eif5a and/or Alox15b/Alox8.

61. The method of claim 54, wherein the sequence modification disrupts a TP53 tumor suppressor gene.

62. The method of claim 54, wherein the nucleic acid sequence information is obtained by long-read sequencing.

63. The method of claim 54, wherein the nucleic acid sequence information is from mRNA.

64. The method of claim 54, wherein the nucleic acid sequence information is obtained by isolating nucleic acid derived from exosomes.

65. The method of claim 64, the method further comprising the step of isolating the exosomes.
